(19) United States
(12) Patent Application Publication
(10) Pub. No.: US 2013/0034515 A1
(43) Pub. Date: Feb. 7, 2013

(54) HAIR CARE COMPOSITIONS

(71) Applicants: Erin A. Stone, Idaho Falls, ID (US); Carmen M. Ramirez, Idaho Falls, ID (US); Rebecca L. Zehntner, Blackfoot, ID (US); David Brock, Idaho Falls, ID (US)

(72) Inventors: Erin A. Stone, Idaho Falls, ID (US); Carmen M. Ramirez, Idaho Falls, ID (US); Rebecca L. Zehntner, Blackfoot, ID (US); David Brock, Idaho Falls, ID (US)

(73) Assignee: Melaleuca, Inc.

(21) Appl. No.: 13/565,095

(22) Filed: Aug. 2, 2012

Related U.S. Application Data
(60) Provisional application No. 61/514,750, filed on Aug. 3, 2011.

(51) Int. Cl. A61K 8/898; A61Q 5/06; A61K 8/92; A61K 8/97; A61Q 5/12; A61Q 5/00; A61Q 5/02

(52) U.S. Cl. 424/70.122; 510/122; 510/121

(57) ABSTRACT

This document provides methods and materials related to hair care. For example, hair care compositions containing one or more quaternized polysiloxanes, one or more zwitterionic surfactants, one or more anionic surfactants, one or more botanical compounds, one or more amino acids, one or more vitamins, or any combination thereof as well as methods for using such hair care compositions are provided.

### CLAIM OF PRIORITY

This application claims priority under 35 USC §119(e) to U.S. Provisional Application Ser. No. 61/514,750, filed on Aug. 3, 2011, the entire contents of which are hereby incorporated by reference.

### BACKGROUND

1. Technical Field

This document relates to hair care and hair care compositions. For example, this document provides hair care compositions that can be applied to hair in a manner that is pleasing to a user and/or in a manner that allows a user to experience a favorable outcome. In some cases, a hair care composition provided herein can be applied to hair in a manner that allows a user to experience minimal fading of a previously applied hair color and/or in a manner that allows a user to experience enhanced thermal protection.

2. Background Information

Many different hair care products are used to treat hair. For example, shampoos are used to clean hair, while styling gels are used to increase hair manageability. Not all hair care products, however, result in healthy hair or in a user's desired goals. In addition, some hair care products can be expensive to produce, thereby limiting the number of users who can benefit from their routine use.

### SUMMARY

This document provides methods and materials related to hair care. For example, this document provides hair care compositions and methods for using hair care compositions. The hair care compositions provided herein can contain one or more quaternized polysiloxanes, one or more zwitterionic surfactants, one or more anionic surfactants, one or more botanical compounds, one or more amino acids, one or more vitamins, or any combination thereof. In some cases, a hair care composition provided herein can lack sulfate. For example, a hair care composition provided herein can be sulfate-free.

The hair care compositions provided herein can provide a desired result (e.g., hair cleaning, hair styling, hair moisturizing, or hair management) in a manner that allows a user to experience minimal fading of a previously applied hair color. For example, users who recently applied hair coloring to their hair can use the hair care compositions provided herein once or repeatedly with minimal fading of a previously applied hair color. In some cases, a hair care composition provided herein can provide a desired result (e.g., hair cleaning, hair styling, hair moisturizing, or hair management) in a manner that allows a user to experience enhanced thermal protection. For example, users can use the hair care products provided herein with enhanced thermal protection such that hair strands exhibit minimal breakage and/or split ends when exposed to heat (e.g., blow drying, curling iron, and/or flat iron).

Having the ability to use the hair care compositions provided herein in a manner that allows a user to experience minimal fading of a previously applied hair color can allow the user to achieve a desired hair care result with more confidence that the hair will retain its color for a sufficient duration. In addition, having the ability to use the hair care compositions provided herein in a manner that allows a user to experience enhanced thermal protection can allow the user to achieve a desired hair care result with more confidence that the hair will remain healthy even when repeatedly exposed to heat (e.g., blow drying, curling iron, and/or flat iron).

In general, one aspect of this document features a hair care composition selected from the group consisting of shampoos, styling gels, aerosol styling sprays, non-aerosol styling sprays, aerosol styling mousses, styling gels, styling pomades, and thermal protection sprays, wherein the hair care composition comprises between about 0.01 percent and about 5 percent of a quaternized polysiloxane. The composition can be a shampoo. The composition can comprise between about 0.01 percent and about 1.5 percent of the quaternized polysiloxane. The composition can comprise between about 0.1 percent and about 1.5 percent of the quaternized polysiloxane. The quaternized polysiloxane can be silicone quaternium-12 or 22. The composition can comprise one or more zwitterionic surfactants. The composition can comprise cocamidopropyl betaine. The composition can comprise one or more anionic surfactants. The composition can comprise sodium cocoyl isethionate, sodium methyl 2-sulfolaurate, disodium 2-sulfolaurate, sodium lauryl sulfoacetate, or a combination thereof. The composition can comprise one or more botanical compounds. The composition can comprise a sunflower seed extract, a sandalwood extract, a Phellodendron amurense bark extract, a barley extract, or a combination thereof. The composition can comprise one or more amino acids. The composition can comprise creatine. The composition can comprise one or more vitamins. The composition can comprise niacinamide, sodium starch octenylsuccinate, calcium pantothenate, maltodextrin, sodium ascorbyl phosphate, tocopheryl acetate, pyridoxine HCl, silica, or a combination thereof. The composition can comprise a zwitterionic surfactant and an anionic surfactant in a ratio from about 1:1 to about 1:2. The composition can comprise a fatty alcohol and a quaternary nitrogen in a ratio from about 1:1 to about 3:1.

In another aspect, this document features a hair care composition comprising between about 0.01 percent and about 5 percent of a quaternized polysiloxane and between about 0.4 percent and about 2 percent of a hydrogenated polydecene. The composition can be a conditioner. The composition can comprise between about 0.01 percent and about 1.5 percent of the quaternized polysiloxane. The composition can comprise between about 0.1 percent and about 1.5 percent of the quaternized polysiloxane. The quaternized polysiloxane can be silicone quaterium-22. The composition can comprise one or more botanical compounds. The composition can comprise a sunflower seed extract, a sandalwood extract, a Phellodendron amurense bark extract, a barley extract, or a combination thereof. The composition can comprise one or more amino acids. The composition can comprise creatine. The composition can comprise one or more vitamins. The composition can comprise niacinamide, sodium starch octenylsuccinate, calcium pantothenate, maltodextrin, sodium ascorbyl phosphate, tocopheryl acetate, pyridoxine HCl, silica, or a combination thereof. The composition can comprise a fatty alcohol and a quaternary nitrogen in a ratio from about 1:1 to about 3:1.

In another aspect, this document features a hair care composition comprising the ingredients set forth in a table selected from the group consisting of Table 1, 2, 3, 4, 5, 6, 7, 8, 9, 10, 11, 12, 13, 14, 15, 16, 17, 18, 19, and 20, wherein each of the ingredients of the hair care composition is present within the hair care composition at a percentage that is within 2 percent of the percentage value set forth in the table. Each of the ingredients of the hair care composition can be present within the hair care composition at a percentage that is within 1 percent of the percentage value set forth in the table. Each of the ingredients of the hair care composition can be present within the hair care composition at a percentage that is within 0.5 percent of the percentage value set forth in the table. Each of the ingredients of the hair care composition can be present within the hair care composition at a percentage that is within 0.25 percent of the percentage value set forth in the table. Each of the ingredients of the hair care composition can be present within the hair care composition at the percentage value set forth in the table.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

This document provides methods and materials related to hair care. For example, this document provides hair care compositions and methods for using hair care compositions. The term “hair care composition” as used herein refers to any product that can be used to clean, treat or style hair. Examples of hair care compositions include, without limitation, shampoos, conditioners, styling gels, aerosol styling sprays, non-aerosol styling sprays, aerosol styling mousses, styling gels, styling pomades, leave-in conditioning sprays, and thermal protection sprays.

The hair care compositions provided herein can be applied to hair in a manner that is pleasing to a user and/or in a manner that allows a user to experience a favorable outcome. In some cases, a hair care composition provided herein can be applied to hair in a manner that allows a user to experience minimal fading of a previously applied hair color and/or in a manner that allows a user to experience enhanced thermal protection. In some cases, a hair care composition provided herein can have superior rheological stability and aesthetic properties.

The hair care compositions provided herein can contain any appropriate combination of the ingredients listed herein. For example, a hair care composition provided herein can contain one or more quaternized polysiloxanes, one or more zwitterionic surfactants, one or more anionic surfactants, one or more botanical compounds, one or more amino acids, one or more vitamins, or any combination thereof.

In some cases, a hair care composition provided herein can lack sulfate. For example, a hair care composition provided herein can be sulfate-free.

### Quaternized Polysiloxanes

A hair care composition provided herein can contain one or more quaternized polysiloxanes. Examples of quaternized polysiloxanes that can be used to make a hair care composition provided herein include, without limitation, silicone quaternium 22 (e.g., Abil T Quat 60), silicone quaternium 12 (Pecosil CA-1240), amodimethicone (e.g., Dow Corning 2-8566 Amino Fluid), bis-cetearyl amodimethicone (e.g., Silsoft AX), bis-amino PEG/PPG-41/3 aminoethyl PG-propyl dimethicone (e.g., Silsoft A-843), PEG-40/PPG-8 methylaminopropyl hydroxypropyl dimethicone copolymer (e.g., Silsoft A+), silicone quaterium 16 (and) undeceth-11 (and) butyloctanol (and) undeceth-5 (e.g., Dow Corning 5-7113 Silicone Quat Microemulsion), and bis-isobutyl/PEG/PPG-20/35/amodimethicone copolymer (e.g., Dow Corning CE 8401 Emulsion). In some cases, a hair care composition provided herein can include silicone quaternium 22.

A hair care composition provided herein can include any appropriate amount of quaternized polysiloxanes. For example, a hair care composition provided herein can contain from about 0.01 percent to about 5 percent (e.g., from about 0.01 percent to about 4 percent, from about 0.01 percent to about 1 percent, from about 0.1 percent to about 4 percent, from about 0.1 percent to about 3 percent, from about 0.1 percent to about 2 percent, from about 0.1 percent to about 1 percent, from about 0.2 percent to about 5 percent, from about 0.3 percent to about 5 percent, from about 0.4 percent to about 5 percent, from about 0.2 percent to about 1 percent, from about 0.2 percent to about 0.8 percent, from about 0.3 percent to about 0.7 percent, or from about 0.4 percent to about 0.6 percent), by weight, of quaternized polysiloxanes. In some cases, a hair care composition provided herein can contain about 0.5 percent quaternized polysiloxanes.

### Zwitterionic Surfactant

A hair care composition provided herein can contain one or more zwitterionic surfactants. Examples of zwitterionic surfactants that can be used to make a hair care composition provided herein include, without limitation, cocamidopropyl betaine, coco betaine (e.g., Dehyton AB), lauryl betaine (e.g., Ampholeen 24), oleyl betaine, stearyl betaine, coco sultaine, and lauryl sultaine. In some cases, a hair care composition provided herein can include cocamidopropyl betaine.

A hair care composition provided herein can include any appropriate amount of zwitterionic surfactants. For example, a hair care composition provided herein can contain from about 1 percent to about 30 percent. (e.g., from about 5 percent to about 30 percent, from about 10 percent to about 30 percent, from about 15 percent to about 30 percent, from about 20 percent to about 30 percent, from about 1 percent to about 25 percent, from about 1 percent to about 20 percent, from about 5 percent to about 25 percent, from about 10 percent to about 25 percent, from about 15 percent to about 25 percent, from about 17 percent to about 23 percent, or from about 19 percent to about 21 percent), by weight, of zwitterionic surfactants. In some cases, a hair care composition provided herein can contain about 20 percent zwitterionic surfactants.

### Anionic Surfactants

A hair care composition provided herein can contain one or more anionic surfactants. Examples of anionic surfactants that can be used to make a hair care composition provided herein include, without limitation, sodium cocoyl isethionate, sodium methyl 2-sulfolaurate, disodium 2-sulfolaurate, sodium lauryl sulfoacetate, ammonium lauryl sulfate (e.g., Standapol A), ammonium laureth sulfate (e.g., Standapol EA), sodium lauryl sulfate (e.g., Standapol WA), sodium laureth sulfate (e.g., Stndapol ES), sodium lauroyl sarcosinate (e.g., Crodasinic LS), cocoyl sarcosine (e.g., Crodasinic C), sodium cocoyl lactylate (e.g., Pationic SCL), sodium cocoyl sarcosinate (e.g., Crodasinic CS). In some cases, a hair care composition provided herein can include a combination of sodium cocoyl isethionate, sodium methyl 2-sulfolaurate, disodium 2-sulfolaurate, and sodium lauryl sulfoacetate.

A hair care composition provided herein can include any appropriate amount of anionic surfactants. For example, a hair care composition provided herein can contain from about 1.5 percent to about 45 percent (e.g., from about 5 percent to about 45 percent, from about 10 percent to about 45 percent, from about 15 percent to about 45 percent, from about 20 percent to about 45 percent, from about 1.5 percent to about 40 percent, from about 1.5 percent to about 35 percent, from about 1.5 percent to about 30 percent, from about 10 percent to about 30 percent, from about 15 percent to about 30 percent, from about 17 percent to about 30 percent, or from about 20 percent to about 25 percent), by weight, of anionic surfactants. In some cases, a hair care composition provided herein can contain about 23 percent anionic surfactants. In some cases, a hair care composition provided herein can contain about 8 percent sodium cocoyl isethionate, sodium methyl 2-sulfolaurate and about 15 percent of a combination of sodium methyl 2-sulfolaurate, disodium 2-sulfolaurate, and sodium lauryl sulfoacetate.

### Botanical Compounds

A hair care composition provided herein can contain one or more botanical compounds. Examples of botanical compounds that can be used to make a hair care composition provided herein include, without limitation, Helianthus annuus (Sunflower) seed extract, Santalum album (Sandalwood) extract, Phellodendron amurense bark extract, Hordeum distichon (Barley) extract, Aloe barbadensis leaf extract, Cassia angustafolia leaf extract, Eucalyptus citriodora leaf extract, Macadamia ternifolia seed extract, Alternifolia leaf extract, palmitoyl grape seed extract, Prunus armeniaca (Apricot) kernel extract, Vitis vinifera (Grape) seed extract, and Yucca aloifolia leaf extract. In some cases, a hair care composition provided herein can include Helianthus annuus (Sunflower) seed extract, Santalum album (Sandalwood) extract, Phellodendron amurense bark extract, and Hordeum distichon (Barley) extract.

A hair care composition provided herein can include any appropriate amount of botanical compounds. For example, a hair care composition provided herein can contain from about 0.01 percent to about 10 percent (e.g., from about 0.05 percent to about 10 percent, from about 0.075 percent to about 10 percent, from about 0.1 percent to about 10 percent, from about 0.01 percent to about 5 percent, from about 0.01 percent to about 2.5 percent, from about 0.01 percent to about 1 percent, from about 0.05 percent to about 5 percent, from about 0.1 percent to about 1 percent, from about 0.2 percent to about 0.5 percent, or from about 0.2 percent to about 0.4 percent), by weight, of botanical compounds. In some cases, a hair care composition provided herein can contain about 0.3 percent botanical compounds. In some cases, a hair care composition provided herein can contain about 0.2 percent of a combination of Helianthus annuus (Sunflower) seed extract and butylene glycol and about 0.1 percent of a combination of Santalum album (Sandalwood) extract, Phellodendron amurense bark Extract, and Hordeum distichon (Barley) extract.

### Amino Acids

A hair care composition provided herein can contain one or more amino acids. Examples of amino acids that can be used to make a hair care composition provided herein include, without limitation, creatine, capryl keratin amino acids, capryl silk amino acids, jojoba amino acids, keratin amino acids, palmitoyl keratin amino acids, palmitoyl silk amino acids, sodium cocoyl amino acids, sodium cocoyl silk amino acids, and sweet almond amino acids. In some cases, a hair care composition provided herein can include creatine.

A hair care composition provided herein can include any appropriate amount of amino acids. For example, a hair care composition provided herein can contain from about 0.001 percent to about 2 percent (e.g., from about 0.005 percent to about 2 percent, from about 0.01 percent to about 2 percent, from about 0.04 percent to about 2 percent, from about 0.001 percent to about 1 percent, from about 0.001 percent to about 0.5 percent, from about 0.001 percent to about 0.1 percent, from about 0.01 percent to about 1 percent, from about 0.025 percent to about 0.1 percent, from about 0.025 percent to about 0.075 percent, or from about 0.04 percent to about 0.06 percent), by weight, of amino acids. In some cases, a hair care composition provided herein can contain about 0.05 percent amino acids.

### Vitamins

A hair care composition provided herein can contain one or more vitamins. Examples of amino acids that can be used to make a hair care composition provided herein include, without limitation, niacinamide, sodium starch octenylsuccinate, calcium pantothenate, maltodextrin, sodium ascorbyl phosphate, tocopheryl acetate, pyridoxine HCl, silica, panthenol (e.g., Pro Vitamin B5), phytantriol, calcium pantothenate (e.g., vitamin B5), vitamin E, and vitamin E esters (e.g., tocopheryl acetate, tocopheryl nocotinate, tocopheryl palmitate, or tocopheryl retinoate). In some cases, a hair care composition provided herein can include niacinamide, sodium starch octenylsuccinate, calcium pantothenate, maltodextrin, sodium ascorbyl phosphate, tocopheryl acetate, pyridoxine HCl, and silica.

A hair care composition provided herein can include any appropriate amount of vitamins. For example, a hair care composition provided herein can contain from about 0.05 percent to about 6.5 percent (e.g., from about 0.1 percent to about 6.5 percent, from about 1 percent to about 6.5 percent, from about 2 percent to about 6.5 percent, from about 0.05 percent to about 6 percent, from about 0.05 percent to about 5 percent, from about 0.05 percent to about 2.5 percent, from about 0.05 percent to about 1 percent, from about 0.1 percent to about 5 percent, from about 0.2 percent to about 2.5 percent, or from about 0.25 percent to about 0.75 percent), by weight, of vitamins. In some cases, a hair care composition provided herein can contain about 0.55 percent vitamins.

### Other Optional Ingredients

The hair care compositions provided herein can contain one or more optional classes of ingredients such pH adjusters, preservatives, solvents, fragrance, and viscosity increasing agents.

### pH Adjustment

The final pH of the undiluted hair care composition provided herein can be between about 4.0 and about 8.5. To obtain such a final pH, the pH of the composition can be adjusted. A pH-adjusting agent can be used to adjust the pH. It will be appreciated that the pH adjustment can be accomplished with any of a wide variety of acids. Common acids that can be used include citric acid, acetic acid, benzoic acid, glycolic acid, lactic acid, malic acid, and sulfuric acid should the composition have a pH too high (e.g., greater than 8.5 before adjustment). Likewise, it will be appreciated that the pH adjustment can be accomplished with any of a wide variety of bases should the composition have a pH too low (e.g., greater than 4.0 before adjustment). Common bases that can be used to lower the pH of these formulations can be potassium hydroxide, potassium carbonate, sodium carbonate, sodium hydroxide, ethanolamine, or triethanolamine.

### Preservatives

To adequately preserve these products and prevent microbial growth, a preservative can be included in a hair care composition provided herein. Acceptable preservatives include, without limitation, benzoic acid, benzyl alcohol, butylparaben, propylparaben, methyparaben, DMDM Hydantoin, potassium benzoate, methylisothiazolinone, methylchloroisothiazolinone, phenoxyethanol, quaterium-8, quaterium-14, quaterium-15, triclosan, zinc pyrithione, and zinc salicylate.

### Solvents

In some cases, one or more solvents can be included in a hair care composition provided herein as an optional ingredient. Example of solvents that can be included in a hair care composition provided herein include, without limitation, butanediol, isoparaffin, cyclomethicone, ethoxyglycol, glycerin, mineral oil, polydimethlysiloxanes, propylene glycol, propanediol, SD alcohol, and shark liver oil.

### Viscosity Modifiers

In some cases, one or more viscosity modifiers can be included in a hair care composition provided herein to help acquire a desired finished product thickness or viscosity. Example of viscosity modifiers that can be included in a hair care composition provided herein include, without limitation, ammonium xylene sulfonate, bentonite, calcium alginate, cocamide DEA, cocamide MEA, dextrin, hectorite, ethylcellulose, guar hydroxypropyltrimonium chloride, hydroxypropyl guar, hydrated silica, lauramide DEA, lauramide MEA, magnesium chloride, methylcellulose, pectin, polyethyleneglycol (PEGs), sodium chloride, sodium stearate, xanthan gum, and zea mays (corn starch).

The invention will be further described in the following examples, which do not limit the scope of the invention described in the claims.

### EXAMPLES

### Example 1

### Shampoo Composition

A moisturizing shampoo composition was prepared by combining the ingredients listed in Table 1 such that the composition contained the indicated percentages by weight. The composition was prepared by heating the water component to a temperature of 160° F. and then adding the remaining ingredients in the order listed in Table 1 while agitating the mixture with sufficient turbulence to acquire a uniform product. Agitation was continued after removing heat until the mixture cooled to room temperature.

TABLE 1
Example of moisturizing shampoo composition.

| | % |
|---|---|
| Ingredient INCI Name | in Formulation |
| Water | 54.030 |
| Cocamidopropyl Betaine | 20.00 |
| Sodium Methyl 2-Sulfolaurate, Disodium 2-Sulfolaurate, | 15.00 |
| and Sodium Lauryl Sulfoacetate | |
| Sodium Cocoyl Isethionate | 8.00 |
| Divinyldimethicone/Dimethicone Copolymer, C12-13 | 0.75 |
| Pareth-23, and C12-13 Pareth-3 | |
| Glycol Distearate | 0.50 |
| Silicone Quaternium22 | 0.25 |
| Guar Hydroxypropyltrimonium Chloride | 0.30 |
| Potassium Sorbate | 0.20 |
| Citric Acid | 0.07 |
| Trisodium Ethylenediamine Disuccinate | 0.05 |
| Methylisothiazolinone and Water | 0.05 |
| Fragrance | 0.80 |

Analysis of this shampoo composition revealed that it effectively cleans hair and that it exhibits aesthetically pleasing characteristics such as lather, rinseability, and a clean/conditioned hair feel.

### Example 2

### Shampoo Composition

An ultra-moisturizing shampoo composition was prepared by combining the ingredients listed in Table 2 such that the composition contained the indicated percentages by weight. Briefly, the composition was prepared by heating the water component to a temperature of 160° F. and then adding the remaining ingredients in the order listed in Table 2 while agitating the mixture with sufficient turbulence to acquire a uniform product. Agitation was continued after removing heat until the mixture cooled to room temperature.

TABLE 2
Example of an ultra-moisturizing shampoo composition.

| Ingredient INCI Name | % in Formulation |
|---|---|
| Water | 53.00 |
| Cocamidopropyl Betaine | 20.00 |
| Sodium Methyl 2-Sulfolaurate, Disodium 2- | 15.00 |
| Sulfolaurate, and Sodium Lauryl Sulfoacetate | |
| Sodium Cocoyl Isethionate | 8.00 |
| Divinyldimethicone/Dimethicone Copolymer, C12-13 | 1.50 |
| Pareth-23, and C12-13 Pareth-3 | |
| Glycol Distearate | 0.50 |
| Silicone Quaternium22 | 0.25 |
| Guar Hydroxypropyltrimonium Chloride | 0.50 |
| Potassium Sorbate | 0.20 |
| Citric Acid | 0.15 |
| Trisodium Ethylenediamine Disuccinate | 0.05 |
| Methylisothiazolinone and Water | 0.05 |
| Fragrance | 0.80 |

Analysis of this shampoo composition revealed that it effectively cleans hair and that it exhibits aesthetically pleasing characteristics such as lather, rinseability, and a clean/conditioned hair feel.

### Example 3

### Shampoo Composition

A volumizing shampoo composition was prepared by combining the ingredients listed in Table 3 such that the composition contained the indicated percentages by weight. Briefly, the composition was prepared by heating the water component to a temperature of 160° F. and then adding the remaining ingredients in the order listed in Table 3 while agitating the mixture with sufficient turbulence to acquire a uniform product. Agitation was continued after removing heat until the mixture cooled to room temperature.

TABLE 3
Example of a volumizing shampoo composition.

| Ingredient INCI Name | % in Formulation |
|---|---|
| Water | 55.525 |
| Cocamidopropyl Betaine | 20.000 |
| Sodium Methyl 2-Sulfolaurate, Disodium | 15.000 |
| 2-Sulfolaurate, and Sodium Lauryl Sulfoacetate | |
| Sodium Cocoyl Isethionate | 8.000 |
| Silicone Quaternium-22 | 0.250 |
| Guar Hydroxypropyltrimonium Chloride | 0.100 |
| Potassium Sorbate | 0.200 |
| Citric Acid | 0.025 |
| Trisodium Ethylenediamine Disuccinate | 0.05 |
| Methylisothiazolinone and Water | 0.05 |
| Fragrance | 0.800 |

Analysis of this shampoo composition revealed that it effectively cleans hair and that it exhibits aesthetically pleasing characteristics such as lather, rinseability, and a clean/conditioned hair feel.

### Example 4

### Shampoo Composition

A detoxifying shampoo composition was prepared by combining the ingredients listed in Table 4 such that the composition contained the indicated percentages by weight. Briefly, the composition was prepared by heating the water component to a temperature of 160° F. and then adding the remaining ingredients in the order listed in Table 4 while agitating the mixture with sufficient turbulence to acquire a uniform product. Agitation was continued after removing heat until the mixture cooled to room temperature.

TABLE 4
Example of a luxury detoxifying shampoo composition.

| | % |
|---|---|
| Ingredient INCI Name | in Formulation |
| Water | 51.825 |
| Cocamidopropyl Betaine | 20.00 |
| Sodium Methyl 2-Sulfolaurate, Disodium 2-Sulfolaurate, | 15.00 |
| and Sodium Lauryl Sulfoacetate | |
| Sodium Cocoyl Isethionate | 8.00 |
| Sodium Polystyrene Sulfonate | 1.00 |
| Vinegar | 1.00 |
| Panthenol | 0.50 |
| Silicone Quaternium-22 | 0.200 |
| Guar Hydroxypropyltrimonium Chloride | 0.100 |
| Potassium Sorbate | 0.20 |
| Helianthus Annuus (Sunflower) Seed Extract and | 0.20 |
| Butylene Glycol | |
| Urtica Diocica (Nettle) Leaf Extract | 0.25 |
| Rosmarinus Officinalis (Rosemary) Leaf Extract | 0.25 |
| Hydrolyzed Soy Protein | 0.200 |
| Santalum Album (Sandalwood) Extract, | 0.10 |
| Phellodendron Amurense Bark Extract, and | |
| Hordeum Distichon (Barley) Extract | |
| Citric Acid | 0.025 |
| Creatine | 0.05 |
| Trisodium Ethylenediamine Disuccinate | 0.05 |
| Methylisothiazolinone and Water | 0.05 |
| Niacinamide, Sodium Starch Octenylsuccinate, Calcium | 0.05 |
| Pantothenate, Maltodextrin, Sodium Ascorbyl Phosphate, | |
| Tocopheryl Acetate, Pyridoxine HCl, and Silica | |
| Fragrance | 0.80 |
| Sodium Hydroxide | 0.15 |

Analysis of this shampoo composition revealed that it effectively cleans hair and that it exhibits aesthetically pleasing characteristics such as lather, rinseability, and a clean hair feel.

### Example 5

### Shampoo Composition

A luxury moisture shampoo composition was prepared by combining the ingredients listed in Table 5 such that the composition contained the indicated percentages by weight. Briefly, the composition was prepared by heating the water component to a temperature of 160° F. and then adding the remaining ingredients in the order listed in Table 5 while agitating the mixture with sufficient turbulence to acquire a uniform product. Agitation was continued after removing heat until the mixture cooled to room temperature.

TABLE 5
Example of a luxury moisture shampoo.

| | % |
|---|---|
| Ingredient INCI Name | in Formulation |
| Water | 52.48 |
| Cocamidopropyl Betaine | 20.00 |
| Sodium Methyl 2-Sulfolaurate, Disodium 2-Sulfolaurate, | 15.00 |
| and Sodium Lauryl Sulfoacetate | |
| Sodium Cocoyl Isethionate | 8.00 |
| Fragrance | 0.80 |
| Divinyldimethicone/Dimethicone Copolymer, C12-13 | 0.75 |
| Pareth-23, and C12-13 Pareth-3 | |
| Glycol Distearate | 0.50 |
| Panthenol | 0.50 |
| Silicone Quaterium-22 | 0.50 |
| Guar Hydroxypropyltrimonium Chloride | 0.30 |
| Potassium Sorbate | 0.20 |
| Helianthus Annuus (Sunflower) Seed Extract and | 0.20 |
| Butylene Glycol | |
| Cocos Nucifera (Coconut) Oil | 0.10 |
| Persea Gratisssima (Avocado) Oil | 0.10 |
| Simmondsia Chinensis (Jojoba) Seed Oil | 0.10 |
| Hydrolyzed Soy Protein | 0.10 |
| Santalum Album (Sandalwood) Extract, | 0.10 |
| Phellodendron Amurense Bark Extract, and | |
| Hordeum Distichon (Barley) Extract | |
| Citric Acid | 0.07 |
| Creatine | 0.05 |
| Trisodium Ethylenedimine Discuccinate | 0.05 |
| Methylisothiazolinone and Water | 0.05 |
| Niacinamide, Sodium Starch Octenylsuccinate, Calcium | 0.05 |
| Pantothenate, Maltodextrin, Sodium Ascorbyl Phosphate, | |
| Tocopheryl Acetate, Pyridoxine HCl, and Silica | |
| Fragrance | 0.80 |

Analysis of this shampoo composition revealed that it effectively cleans hair and that it exhibits aesthetically pleasing characteristics such as lather, rinseability, and a clean/conditioned hair feel.

### Example 6

### Shampoo Composition

A luxury ultra moisture shampoo composition was prepared by combining the ingredients listed in Table 6 such that the composition contained the indicated percentages by weight. Briefly, the composition was prepared by heating the water component to a temperature of 160° F. and then adding the remaining ingredients in the order listed in Table 6 while agitating the mixture with sufficient turbulence to acquire a uniform product. Agitation was continued after removing heat until the mixture cooled to room temperature.

TABLE 6
Example of a luxury ultra moisture shampoo

| | % in |
|---|---|
| Ingredient INCI Name | Formulation |
| Water | 51.45 |
| Cocamidopropyl Betaine | 20.00 |
| Sodium Methyl 2-Sulfolaurate, Disodium 2-Sulfolaurate, | 15.00 |
| and Sodium lauryl Sulfoacetate | |
| Sodium Cocoyl Isethionate | 8.00 |
| Divinyldimethicone/Dimethicone Copolymer, C12-13 | 1.50 |
| Pareth-23, and C12-13 Pareth-3 | |
| Glycol Distearate | 0.50 |
| Panthenol | 0.50 |
| Silicone Quaternium-22 | 0.50 |
| Guar Hydroxypropyltrimonium Chloride | 0.50 |
| Potassium Sorbate | 0.20 |
| Helianthus Annuus (Sunflower) Seed Extract and Butylene | 0.20 |
| Glycol | |
| Cocos Nucifera (Coconut) Oil | 0.10 |
| Persea Gratisssima (Avocado) Oil | 0.10 |
| Simmondsia Chinensis (Jojoba) Seed Oil | 0.10 |
| Hydrolyzed Soy Protein | 0.10 |
| Santalum Album (Sandalwood) Extract, | 0.10 |
| PhellodendronAmurense Bark Extract, and | |
| Hordeum Distichon (Barley)Extract | |
| Citric Acid | 0.07 |
| Creatine | 0.05 |
| Trisodium Ethylenediamine Disuccinate | 0.05 |
| Methylisothiazolinone and Water | 0.05 |
| Niacinamide, Sodium Starch Octenylsuccinate, Calcium | 0.05 |
| Pantothenate, Maltodextrin, Sodium Ascorbyl Phosphate, | |
| Tocopheryl Acetate, Pyridoxine HCl, and Silica | |
| Fragrance | 0.80 |

Analysis of this shampoo composition revealed that it effectively cleans hair and that it exhibits aesthetically pleasing characteristics such as lather, rinseability, and a clean/conditioned hair feel.

### Example 7

### Shampoo Composition

A luxury volume shampoo composition was prepared by combining the ingredients listed in Table 7 such that the composition contained the indicated percentages by weight. Briefly, the composition was prepared by heating the water component to a temperature of 160° F. and then adding the remaining ingredients in the order listed in Table 7 while agitating the mixture with sufficient turbulence to acquire a uniform product. Agitation was continued after removing heat until the mixture cooled to room temperature.

TABLE 7
Example of luxury volume shampoo.

| | % |
|---|---|
| Ingredient INCI Name | in Formulation |
| Water | 53.675 |
| Cocamidopropyl Betaine | 20.000 |
| Sodium Methyl 2-Sulfolaurate, Disodium 2-Sulfolaurate, | 15.000 |
| and Sodium Lauryl Sulfoacetate | |
| Sodium Cocoyl Isethionate | 8.000 |
| Panthenol | 0.500 |
| Silicone Quaternium-22 | 0.500 |
| Water and Urtica Dioica (Nettle) Extract | 0.2500 |
| Water and Rosmarinus Officinalis (Rosemary) Leaf | 0.2500 |
| Extract | |
| Guar Hydroxypropyltrimonium Chloride | 0.100 |
| Potassium Sorbate | 0.200 |
| Helianthus Annuus (Sunflower) Seed Extract and | 0.200 |
| Butylene Glycol | |
| Simmondsia Chinensis (Jojoba) Seed Oil | 0.100 |
| Hydrolyzed Soy Protein | 0.100 |
| Santalum Album (Sandalwood) Extract, | 0.100 |
| PhellodendronAmurense Bark Extract, and | |
| Hordeum Distichon (Barley) Extract | |
| Citric Acid | 0.025 |
| Creatine | 0.05 |
| Trisodium Ethylenediamine Disuccinate | 0.05 |
| Methylisothiazolinone and Water | 0.05 |
| Niacinamide, Sodium Starch Octenylsuccinate, Calcium | 0.05 |
| Pantothenate, Maltodextrin, Sodium Ascorbyl Phosphate, | |
| Tocopheryl Acetate, Pyridoxine HCl, and Silica | |
| Fragrance | 0.800 |

Analysis of this shampoo composition revealed that it effectively cleans hair and that it exhibits aesthetically pleasing characteristics such as lather, rinseability, and a clean/conditioned hair feel.

### Example 8

### Conditioner Composition

A moisturize composition was prepared by combining the ingredients listed in Table 8 such that the composition contained the indicated percentages by weight. Briefly, the composition was prepared by heating the water component to a temperature of 170° F. and then adding the remaining ingredients in the order listed in Table 8 while agitating the mixture with sufficient turbulence to acquire a uniform product. Agitation was continued after removing heat until the mixture cooled to room temperature.

TABLE 8
Example of a moisturize conditioner.

| | % |
|---|---|
| Ingredient INCI Name | in Formulation |
| Water | 85.615 |
| Quaternium-91, Cetrimonium Methosulfate, and Cetearyl | 2.250 |
| Alcohol | |
| Stearyl Alcohol | 2.130 |
| Distearoylethyl Dimonium Chloride and Cetearyl | 1.350 |
| Alcohol | |
| Cetyl Alcohol | 1.280 |
| Hydrogenated Polydecene | 1.250 |
| Cetearyl Alcohol | 0.900 |
| Glycerin | 1.000 |
| Propanediol | 1.000 |
| Dicaprylyl Carbonate | 1.000 |
| Fragrance | 0.800 |
| Quaternium-80 | 0.250 |
| Silicone Quaternium-22 | 0.250 |
| Stearamidopropyl Dimethylamine | 0.500 |
| Sorbic Acid | 0.150 |
| Guar Hydroxypropyltrimonium Chloride | 0.150 |
| Tetrasodium EDTA | 0.050 |
| Methylisothiazolinone and Water | 0.050 |
| Citric Acid | 0.025 |

Analysis of this conditioner composition revealed that it effectively conditions hair and that it exhibits aesthetically pleasing characteristics such as rinseability, wet detangling, and anti-static benefits.

### Example 9

### Conditioner Composition

An ultra-moisturizing composition was prepared by combining the ingredients listed in Table 9 such that the composition contained the indicated percentages by weight. Briefly, the composition was prepared by heating the water component to a temperature of 170° F. and then adding the remaining ingredients in the order listed in Table 9 while agitating the mixture with sufficient turbulence to acquire a uniform product. Agitation was continued after removing heat until the mixture cooled to room temperature.

TABLE 9
Example of ultra-moisturizing conditioner.

| Ingredient INCI Name | % in Formulation |
|---|---|
| Water | 81.170 |
| Brassicamidopropyl Dimethylamine | 3.500 |
| Stearyl Alcohol | 4.050 |
| Distearoylethyl Dimonium Chloride and Cetearyl | 1.350 |
| Alcohol | |
| Cetyl Alcohol | 2.430 |
| Hydrogenated Polydecene | 1.000 |
| Cetearyl Alcohol | 0.900 |
| Cetrimonium Chloride | 1.000 |
| Cyclopentasiloxane and Dimethicone Crosspolymer | 0.850 |
| Phenoxyethanol | 0.800 |
| Fragrance | 0.800 |
| Quaternium-80 | 0.250 |
| Silicone Quaternium-22 | 0.250 |
| Cyclopentasiloxane | 0.500 |
| Lactic Acid | 0.350 |
| Sorbic Acid | 0.150 |
| Guar Hydroxypropyltrimonium Chloride | 0.250 |
| Tetrasodium EDTA | 0.100 |
| Citric Acid | 0.300 |

Analysis of this conditioner composition revealed that it effectively conditions hair and that it exhibits aesthetically pleasing characteristics such as rinseability, wet detangling, and anti-static benefits.

### Example 10

### Conditioner Composition

A volumizing composition was prepared by combining the ingredients listed in Table 10 such that the composition contained the indicated percentages by weight. Briefly, the composition was prepared by heating the water component to a temperature of 170° F. and then adding the remaining ingredients in the order listed in Table 10 while agitating the mixture with sufficient turbulence to acquire a uniform product. Agitation was continued after removing heat until the mixture cooled to room temperature.

TABLE 10
Example of volumizing conditioner.

| | % in |
|---|---|
| Ingredient INCI Name | Formulation |
| Water | 88.300 |
| Behentrimonium Methosulfate and Cetearyl Alcohol | 2.500 |
| Stearyl Alcohol | 1.800 |
| Distearoylethyl Dimonium Chloride and Cetearyl Alcohol | 1.350 |
| Cetyl Alcohol | 0.900 |
| Hydrogenated Polydecene | 0.500 |
| Cetearyl Alcohol | 0.900 |
| Propanediol | 1.000 |
| PPG-3 Benzyl Ether Ethylhexanoate | 0.500 |
| Dicaprylyl Carbonate | 0.250 |
| Fragrance | 0.850 |
| Silicone Quaternium-22 | 0.250 |
| Stearamidopropyl Dimethylamine | 0.500 |
| Sorbic Acid | 0.200 |
| Guar Hydroxypropyltrimonium Chloride | 0.100 |
| Tetrasodium EDTA | 0.050 |
| Methylisothiazolinone and Water | 0.050 |

Analysis of this conditioner composition revealed that it effectively conditions hair and that it exhibits aesthetically pleasing characteristics such as rinseability, wet detangling, and anti-static benefits.

### Example 11

### Conditioner Composition

A luxury moisturize crème rinse composition was prepared by combining the ingredients listed in Table 11 such that the composition contained the indicated percentages by weight. Briefly, the composition was prepared by heating the water component to a temperature of 170° F. and then adding the remaining ingredients in the order listed in Table 11 while agitating the mixture with sufficient turbulence to acquire a uniform product. Agitation was continued after removing heat until the mixture cooled to room temperature.

TABLE 11
Example of luxury moisture crème rinse.

| | % in |
|---|---|
| Ingredient INCI Name | Formulation |
| Water | 81.652 |
| Quaternium-91, Cetrimonium Methosulfate, and Cetearyl | 2.500 |
| Alcohol | |
| Stearyl Alcohol | 2.133 |
| Distearoylethyl Dimonium Chloride and Cetearyl Alcohol | 1.500 |
| Cetyl Alcohol | 1.290 |
| Hydrogenated Polydecene | 1.250 |
| Cetearyl Alcohol | 1.000 |
| Glycerin | 1.000 |
| Propanediol | 1.000 |
| PPG-3 Benzyl Ether Ethylhexanoate | 1.000 |
| Dicaprylyl Carbonate | 1.000 |
| Fragrance | 0.800 |
| Quaternium-80 | 0.500 |
| Silicone Quaternium-22 | 0.500 |
| Stearamidopropyl Dimethylamine | 0.500 |
| Panthenol | 0.500 |
| Santalum Album (Sandalwood) Extract, | 0.500 |
| Phellodendron Amurense Bark Extract, and | |
| Hordeum Distichon (Barley) Extract | |
| Hydrolyzed Soy Protein | 0.200 |
| Helianthus Annuus (Sunflower) Seed Extract and Butylene | 0.200 |
| Glycol | |
| Sorbic Acid | 0.150 |
| Guar Hydroxypropyltrimonium Chloride | 0.150 |
| Cocos Nucifera (Coconut) Oil | 0.100 |
| Persea Gratisssima (Avocado) Oil | 0.100 |
| Simmondsia Chinensis (Jojoba) Seed Oil | 0.100 |
| Phytantriol | 0.100 |
| Creatine | 0.050 |
| Tetrasodium EDTA | 0.050 |
| Methylisothiazolinone and Water | 0.050 |
| Niacinamide, Sodium Starch Octenylsuccinate, Calcium | 0.050 |
| Pantothenate, Maltodextrin, Sodium Ascorbyl Phosphate, | |
| Tocopheryl Acetate, Pyridoxine HCl, and Silica | |

Analysis of this conditioner composition revealed that it effectively conditions hair and that it exhibits aesthetically pleasing characteristics such as rinseability, wet detangling, and anti-static benefits.

### Example 12

### Conditioner Composition

A luxury ultra moisturize crème rinse composition was prepared by combining the ingredients listed in Table 12 such that the composition contained the indicated percentages by weight. Briefly, the composition was prepared by heating the water component to a temperature of 170° F. and then adding the remaining ingredients in the order listed in Table 12 while agitating the mixture with sufficient turbulence to acquire a uniform product. Agitation was continued after removing heat until the mixture cooled to room temperature.

TABLE 12
Example of luxury ultra moisture crème rinse.

| Ingredient INCI Name | % in Formulation |
|---|---|
| Water | 76.860 |
| Brassicamidopropyl Dimethylamine | 3.500 |
| Stearyl Alcohol | 4.050 |
| Distearoylethyl Dimonium Chloride and Cetearyl | 1.500 |
| Alcohol | |
| Cetyl Alcohol | 2.43 |
| Hydrogenated Polydecene | 1.000 |
| Cetearyl Alcohol | 1.000 |
| Cetrimonium Chloride | 1.000 |
| Cyclopentasiloxane and Dimethicone Crosspolymer | 0.850 |
| Phenoxyethanol | 0.800 |
| Fragrance | 0.800 |
| Quaternium-80 | 0.500 |
| Silicone Quaternium-22 | 0.500 |
| Stearamidopropyl Dimethylamine | 0.500 |
| Panthenol | 0.500 |
| Santalum Album (Sandalwood) Extract, | 0.500 |
| Phellodendron Amurense Bark Extract, and | |
| Hordeum Distichon (Barley) Extract | |
| Cyclopentasiloxane | 0.500 |
| Hydrolyzed Soy Protein | 0.200 |
| Helianthus Annuus (Sunflower) Seed Extract and | 0.200 |
| Butylene Glycol | |
| Lactic Acid | 0.350 |
| Sorbic Acid | 0.150 |
| Guar Hydroxypropyltrimonium Chloride | 0.250 |
| Cocos Nucifera (Coconut) Oil | 0.500 |
| Persea Gratisssima (Avocado) Oil | 0.500 |
| Simmondsia Chinensis (Jojoba) Seed Oil | 0.500 |
| Phytantriol | 0.100 |
| Creatine | 0.010 |
| Tetrasodium EDTA | 0.100 |

Analysis of this conditioner composition revealed that it effectively conditions hair and that it exhibits aesthetically pleasing characteristics such as rinseability, wet detangling, and anti-static benefits.

### Example 13

### Conditioner Composition

A luxury volumizing crème rinse composition was prepared by combining the ingredients listed in Table 13 such that the composition contained the indicated percentages by weight. Briefly, the composition was prepared by heating the water component to a temperature of 170° F. and then adding the remaining ingredients in the order listed in Table 13 while agitating the mixture with sufficient turbulence to acquire a uniform product. Agitation was continued after removing heat until the mixture cooled to room temperature.

TABLE 13
Example of luxury volume creme rinse.

| | % in |
|---|---|
| Ingredient INCI Name | Formulation |
| Water | 81.652 |
| Behentrimonium Methosulfate and Cetearyl Alcohol | 2.500 |
| Stearyl Alcohol | 1.800 |
| Distearoylethyl Dimonium Chloride and Cetearyl | 1.500 |
| Alcohol | |
| Cetyl Alcohol | 0.900 |
| Hydrogenated Polydecene | 0.500 |
| Cetearyl Alcohol | 1.000 |
| Propanediol | 1.000 |
| PPG-3 Benzyl Ether Ethylhexanoate | 0.500 |
| Dicaprylyl Carbonate | 0.250 |
| Fragrance | 0.850 |
| Quaternium-80 | 0.500 |
| Silicone Quaternium-22 | 0.500 |
| Stearamidopropyl Dimethylamine | 0.500 |
| Panthenol | 0.250 |
| Santalum Album (Sandalwood) Extract, Phellodendron | 0.500 |
| Amurense Bark Extract, and Hordeum Distichon (Barley) | |
| Extract | |
| Hydrolyzed Soy Protein | 0.200 |
| Helianthus Annuus (Sunflower) Seed Extract and | 0.200 |
| Butylene Glycol | |
| Sorbic Acid | 0.200 |
| Guar Hydroxypropyltrimonium Chloride | 0.100 |
| Water and Urtica Dioica (Nettle) Extract | 0.100 |
| Water and Rosmarinus Officinalis (Rosemary) Extract | 0.100 |
| Phytantriol | 0.100 |
| Creatine | 0.100 |
| Tetrasodium EDTA | 0.050 |
| Methylisothiazolinone and Water | 0.050 |
| Niacinamide, Sodium Starch Octenylsuccinate, Calcium | 0.050 |
| Pantothenate, Maltodextrin, Sodium Ascorbyl Phosphate, | |
| Tocopheryl Acetate, Pyridoxine HCl, and Silica | |

Analysis of this conditioner composition revealed that it effectively conditions hair and that it exhibits aesthetically pleasing characteristics such as rinseability, wet detangling, and anti-static benefits.

### Example 14

### Repair Hair Masque Composition

A luxury repair hair masque composition was prepared by combining the ingredients listed in Table 14 such that the composition contained the indicated percentages by weight. Briefly, the composition was prepared by heating the water component to a temperature of 170° F. and then adding the remaining ingredients in the order listed in Table 14 while agitating the mixture with sufficient turbulence to acquire a uniform product. Agitation was continued after removing heat until the mixture cooled to room temperature.

TABLE 14
Example of luxury repair hair masque.

| | % in |
|---|---|
| Ingredient INCI Name | Formulation |
| Water | 66.900 |
| Brassicamidopropyl Dimethylamine | 3.000 |
| Stearyl Alcohol | 5.000 |
| Distearoylethyl Dimonium Chloride and Cetearyl | 0.250 |
| Alcohol | |
| Cetyl Alcohol | 3.500 |
| Hydrogenated Polydecene | 1.000 |
| Quaternium-91, Cetrimonium Methosulfate, and Cetearyl | 2.000 |
| Alcohol | |
| Glycerin | 3.000 |
| Dimethicone | 1.500 |
| Cyclopentasiloxane | 1.500 |
| Butyrospermum Parkii (Shea Butter) | 1.250 |
| Cetearyl Alcohol | 1.000 |
| PPG-3 Benzyl Ether Ethylhexanoate | 1.000 |
| Amodimethicone, Trideceth-12, and Cetrimonium | 0.500 |
| Chloride | |
| Cyclopentasiloxane and Dimethicone Crosspolymer | 0.500 |
| Fragrance | 0.800 |
| Quaternium-80 | 0.500 |
| Silicone Quaternium-22 | 1.500 |
| Stearamidopropyl Dimethylamine | 0.500 |
| Panthenol | 1.000 |
| Santalum Album (Sandalwood) Extract, Phellodendron | 1.000 |
| Amurense Bark Extract, and Hordeum Distichon (Barley) | |
| Extract | |
| Hydrolyzed Soy Protein | 0.200 |
| Helianthus Annuus (Sunflower) Seed Extract and | 0.200 |
| Butylene Glycol | |
| Lactic Acid | 0.350 |
| Sorbic Acid | 0.200 |
| Guar Hydroxypropyltrimonium Chloride | 0.250 |
| Cocos Nucifera (Coconut) Oil | 0.250 |
| Persea Gratisssima (Avocado) Oil | 0.250 |
| Simmondsia Chinensis (Jojoba) Seed Oil | 0.250 |
| Phytantriol | 0.100 |
| Creatine | 0.100 |
| Tetrasodium EDTA | 0.100 |
| Methylisothiazolinone and Water | 0.050 |
| Niacinamide, Sodium Starch Octenylsuccinate, Calcium | 0.050 |
| Pantothenate, Maltodextrin, Sodium Ascorbyl Phosphate, | |
| Tocopheryl Acetate, Pyridoxine HCl, and Silica | |
| Citric Acid | 0.500 |

Analysis of this hair masque composition revealed that it effectively deep conditions hair and that it exhibits aesthetically pleasing characteristics such as rinseability, wet detangling, smooth texture of dry treated hair and anti-static benefits.

### Example 15

### Leave-In Detangling Spray Composition

A leave-in detangling spray composition was prepared by combining the ingredients listed in Table 15 such that the composition contained the indicated percentages by weight. Briefly, the composition was prepared at ambient temperature by adding the ingredients in the order listed in Table 15 to the bulk water phase while agitating the mixture with sufficient turbulence to acquire a uniform product.

TABLE 15
Example of luxury leave-in detangling and smoothing spray.

| | % in |
|---|---|
| Ingredient INCI Name | Formulation |
| Water | 94.660 |
| Cetrimonium Chloride | 1.60 |
| Bis(C13-15 Alkoxy) PG-Amodimethicone | 1.20 |
| Caprylyl Glycol and 1,2-Hexandediol | 0.50 |
| Phenoxyethanol | 0.40 |
| Fragrance | 0.40 |
| Panthenol | 0.20 |
| Silicone Quaternium-22 | 0.40 |
| Polysorbate 80 | 0.20 |
| Helianthus Annuus (Sunflower) Seed Extract and | 0.10 |
| Butylene Glycol | |
| Hydrolyzed Soy Protein | 0.10 |
| Santalum Album (Sandalwood) Extract, Phellodendron | 0.01 |
| Amurense Bark Extract, and Hordeum Distichon (Barley) | |
| Extract | |
| Creatine | 0.01 |
| Aminomethyl Propanol | 0.02 |
| Disodium EDTA | 0.10 |
| Niacinamide, Sodium Starch Octenylsuccinate, Calcium | 0.10 |
| Pantothenate, Maltodextrin, Sodium Ascorbyl Phosphate, | |
| Tocopheryl Acetate, Pyridoxine HCl, and Silica | |

Analysis of this leave-in detangling spray composition revealed that it effectively conditions hair and that it exhibits aesthetically pleasing characteristics such as ease of wet detangling and anti-static benefits.

### Example 16

### Heat Protection Spray Composition

A heat protection spray composition was prepared by combining the ingredients listed in Table 16 such that the composition contained the indicated percentages by weight. Briefly, the composition was prepared at ambient temperature by adding the ingredients in the order listed in Table 17 to the bulk water phase while agitating the mixture with sufficient turbulence to acquire a uniform product.

TABLE 16
Example of luxury hot iron prep spray.

| | % in |
|---|---|
| Ingredient INCI Name | Formulation |
| Water | 89.93 |
| Cetrimonium Chloride | 2.500 |
| Glycerin | 2.000 |
| Sodium Cocoyl Amino Acids, Potassium Dimethicone | 1.000 |
| PEG-7 Panthenyl Phosphate | |
| Sodium Laneth-40 Maleate/Styrene Sulfonate Copolymer | 1.000 |
| Polysorbate 20 | 0.750 |
| Phenoxyethanol | 0.600 |
| PEG-12 Dimethicone | 0.500 |
| Panthenol | 0.500 |
| Fragrance | 0.500 |
| Phytantriol | 0.100 |
| Silicone Quaternium-22 | 0.05 |
| Potassium Sorbate | 0.20 |
| Aminomethyl Propanol | 0.03 |
| Helianthus Annuus (Sunflower) Seed Extract and | 0.01 |
| Butylene Glycol | |
| Hydrolyzed Soy Protein | 0.10 |
| Santalum Album (Sandalwood) Extract, Phellodendron | 0.01 |
| Amurense Bark Extract, and Hordeum Distichon (Barley) | |
| Extract | |
| Creatine | 0.01 |
| Disodium EDTA | 0.20 |
| Niacinamide, Sodium Starch Octenylsuccinate, Calcium | 0.01 |
| Pantothenate, Maltodextrin, Sodium Ascorbyl Phosphate, | |
| Tocopheryl Acetate, Pyridoxine HCl, and Silica | |

Analysis of this hot iron prep spray composition revealed that it effectively protects and conditions hair during the hot styling process utilizing a curling iron or heated styling appliance and that it exhibits aesthetically pleasing characteristics such as smooth/soft hair in the dry state, shine, and anti-static benefits.

### Example 17

### Non-Aerosol Hair Spray Composition

A non-aerosol hair spray composition was prepared by combining the ingredients listed in Table 17 such that the composition contained the indicated percentages by weight. Briefly, the composition was prepared at ambient temperature by adding the ingredients in the order listed in Table 17 to the bulk water phase while agitating the mixture with sufficient turbulence to acquire a uniform product. Agitation was continued after until the mixture was uniform and homogeneous.

TABLE 17
Example of luxury classic hold finishing spray.

| | % in |
|---|---|
| Ingredient INCI Name | Formulation |
| SD Alcohol 40-B | 55.00 |
| Water | 32.55 |
| Acrylates/Hydroxyesters Acrylates Copolymer | 8.70 |
| VA/Crotonates/Vinyl Neodecanoate Copolymer | 2.00 |
| Aminomethyl Propanol | 0.65 |
| Fragrance | 0.40 |
| Polyquaternium-69 | 0.20 |
| Lauryl Pyrrolidone | 0.20 |
| Panthenol | 0.10 |
| Silicone Quaternium-22 | 0.01 |
| PEG-12 Dimethicone | 0.10 |
| Helianthus Annuus (Sunflower) Seed Extract and | 0.01 |
| Butylene Glycol | |
| Water, Butylene Glycol, and Vitis Vinifera (Grape) Leaf | 0.01 |
| Extract | |
| Water (and) Urtica Dioica (Nettle) Extract | 0.01 |
| Rosmarinus Officinalis (Rosemary) Leaf Extract | 0.01 |
| Hydrolyzed Soy Protein | 0.01 |
| Santalum Album (Sandalwood) Extract, Phellodendron | 0.01 |
| Amurense Bark Extract, and Hordeum Distichon (Barley) | |
| Extract | |
| Creatine | 0.01 |
| Phytantriol | 0.01 |
| Niacinamide, Sodium Starch Octenylsuccinate, Calcium | 0.01 |
| Pantothenate, Maltodextrin, Sodium Ascorbyl Phosphate, | |
| Tocopheryl Acetate, Pyridoxine HCl, and Silica | |

Analysis of this finishing spray composition revealed that it effectively styles hair and that it exhibits aesthetically pleasing characteristics such as curl retention and maintenance of desired hair style throughout the day.

### Example 18

### Styling Gel Composition

A styling gel composition was prepared by combining the ingredients listed in Table 18 such that the composition contained the indicated percentages by weight. Briefly, the composition was prepared at ambient temperature by adding the ingredients in the order listed in Table 18 to the bulk water phase while agitating the mixture with sufficient turbulence to acquire a uniform product. Agitation was continued after until the mixture was uniform and homogeneous.

TABLE 18
Example of luxury defining gel.

| | % in |
|---|---|
| Ingredient INCI Name | Formulation |
| Water | 86.90 |
| AMP-Acrylates/Alkyl Methacrylate Copolymer | 3.90 |
| Polyacrylamide-2 Crosspolymer | 3.60 |
| Aminomethyl Propanol | 1.20 |
| Acrylates/C10-30 Alkyl Acrylate Crosspolymer | 0.70 |
| PVP | 0.60 |
| Polyquaternium-69 | 0.50 |
| PEG-40 Hydrogenated Castor Oil | 0.50 |
| Phenoxyethanol | 0.50 |
| Panthenol | 0.50 |
| Glycerin and Glyceryl Acrylates/Acrylic Acid Copolymer | 0.30 |
| Glycerin | 0.20 |
| Fragrance | 0.20 |
| Tetrasodium EDTA | 0.10 |
| PEG-12 Dimethicone | 0.10 |
| Silicone Quaternium-22 | 0.10 |
| Phytantriol | 0.01 |
| Helianthus Annuus (Sunflower) Seed Extract and Butylene | 0.01 |
| Glycol | |
| Hydrolyzed Soy Protein | 0.01 |
| Santalum Album (Sandalwood) Extract, Phellodendron | 0.01 |
| Amurense Bark Extract, and Hordeum Distichon (Barley) | |
| Extract | |
| Creatine | 0.01 |
| Methylisothiazolinone and Water | 0.05 |
| Niacinamide, Sodium Starch Octenylsuccinate, Calcium | 0.01 |
| Pantothenate, Maltodextrin, Sodium Ascorbyl Phosphate, | |
| Tocopheryl Acetate, Pyridoxine HCl, and Silica | |

Analysis of this styling gel composition revealed that it effectively styles hair by enabling the user to achieve their desired hair style and that it exhibits aesthetically pleasing characteristics such as maintenance of hair style throughout the day

### Example 19

### Styling Crème Composition

A styling crème composition was prepared by combining the ingredients listed in Table 19 such that the composition contained the indicated percentages by weight. Briefly, the composition was prepared at ambient temperature by adding the ingredients in the order listed in Table 19 to the bulk water phase while agitating the mixture with sufficient turbulence to acquire a uniform product. Agitation was continued after until the mixture was uniform and homogeneous.

TABLE 19
Example of luxury soft hold styling creme.

| | % in |
|---|---|
| Ingredient INCI Name | Formulation |
| Water | 89.987 |
| Sorbitol | 1.500 |
| Hydrogenated Polydecene | 1.500 |
| Dehydroxanthan Gum | 1.500 |
| Phenoxyethanol | 0.700 |
| Polyquaternium-69 | 0.500 |
| PPG-3 Benzyl Ether Ethylhexanoate | 0.500 |
| Fragrance | 0.300 |
| Silicone Quaternium-22 | 0.500 |
| Polyquaternium-4/Hydroxypropyl Starch Copolymer | 0.200 |
| Sodium Polystyrene Sulfonate | 0.200 |
| Panthenol | 0.500 |
| Santalum Album (Sandalwood) Extract, Phellodendron | 1.000 |
| Amurense Bark Extract, and Hordeum Distichon (Barley) | |
| Extract | |
| Hydrolyzed Soy Protein | 0.200 |
| Helianthus Annuus (Sunflower) Seed Extract and | 0.100 |
| Butylene Glycol | |
| Persea Gratisssima (Avocado) Oil | 0.100 |
| Simmondsia Chinensis (Jojoba) Seed Oil | 0.100 |
| Phytantriol | 0.100 |
| Creatine | 0.100 |
| Trisodium Ethylenediamine Disuccinate | 0.050 |
| Methylisothiazolinone and Water | 0.050 |
| Niacinamide, Sodium Starch Octenylsuccinate, Calcium | 0.050 |
| Pantothenate, Maltodextrin, Sodium Ascorbyl Phosphate, | |
| Tocopheryl Acetate, Pyridoxine HCl, and Silica | |
| Citric Acid | 0.013 |

Analysis of this styling crème composition revealed that it effectively styles and conditions hair and that it exhibits aesthetically pleasing characteristics such as smooth soft hair feel, anti-static benefits, and maintenance of style throughout the day.

### Example 20

### Luxury Texturizing Pomade Composition

A luxury texturizing pomade composition was prepared by combining the ingredients listed in Table 20 such that the composition contained the indicated percentages by weight. Briefly, the composition was prepared by heating the water component to a temperature of 160° F. and then adding the remaining ingredients in the order listed in Table 7 while agitating the mixture with sufficient turbulence to acquire a uniform product. Agitation was continued after removing heat until the mixture cooled to room temperature.

TABLE 20
Luxury Texturizing Pomade

| | % in |
|---|---|
| Ingredient INCI Name | Formulation |
| Water | 47.24 |
| Isoceteth-20 | 20.00 |
| PPG-3 Benzyl Ether Ethylhexanoate | 10.00 |
| Oleth-2 | 6.00 |
| Propanediol | 5.00 |
| Polyurethane-14 and AMP-Acrylates Copolymer | 5.00 |
| Sorbitol | 1.000 |
| Sodium Polystyrene Sulfonate | 1.000 |
| PVP | 1.000 |
| Fragrance | 1.000 |
| Panthenol | 0.10 |
| Silicone Quaternium-22 | 0.50 |
| Polyquaternium-4/Hydroxypropyl Starch Copolymer | 0.200 |
| Helianthus Annuus (Sunflower) Seed Extract and Butylene | 0.20 |
| Glycol | |
| Cocos Nucifera (Coconut) Oil | 0.10 |
| Persea Gratisssima (Avocado) Oil | 0.10 |
| Simmondsia Chinensis (Jojoba) Seed Oil | 0.10 |
| Hydrolyzed Soy Protein | 0.20 |
| Santalum Album (Sandalwood) Extract, Phellodendron | 1.00 |
| Amurense Bark Extract, and Hordeum Distichon (Barley) | |
| Extract | |
| Phytantriol | 0.01 |
| Creatine | 0.10 |
| Tetrasodium EDTA | 0.05 |
| Methylisothiazolinone and Water | 0.05 |
| Niacinamide, Sodium Starch Octenylsuccinate, Calcium | 0.05 |
| Pantothenate, Maltodextrin, Sodium Ascorbyl Phosphate, | |
| Tocopheryl Acetate, Pyridoxine HCl, and Silica | |

Analysis of this luxury texturizing pomade composition revealed that it effectively styles hair by enabling a user to acquire their desired style and that it exhibits aesthetically pleasing characteristics such as anti-static benefits and maintenance of style throughout the day.

### Example 21

### Shampoo Composition

A moisturizing shampoo composition can be prepared by combining the ingredients listed in Table 21 such that the composition contains the indicated ingredients in the disclosed ranges (in percentages by weight). In some cases, the Silicone Quaternium-22 of the composition shown in Table 21 can be partially or fully replaced with 0.30-1.0 weight percent of Silicone Quaternium-12.

TABLE 21
Example of moisturizing shampoo composition.

| Ingredient INCI Name | % in Formulation |
|---|---|
| Water | Balance |
| Cocamidopropyl Betaine | 10.00-30.00 |
| Sodium Methyl 2-Sulfolaurate, Disodium | 10.00-30.00 |
| 2-Sulfolaurate, and Sodium Lauryl Sulfoacetate | |
| Sodium Cocoyl Isethionate | 3.00-10.00 |
| Divinyldimethicone/Dimethicone Copolymer, C12-13 | 0.30-1.0 |
| Pareth-23, and C12-13 Pareth-3 | |
| Glycol Distearate | 0.30-1.0 |
| Silicone Quaternium-22 | 0.0.1-0.30 |
| Guar Hydroxypropyltrimonium Chloride | 0.30-1.0 |
| Potassium Sorbate | 0.10-0.30 |
| Citric Acid | 0.00-0.10 |
| Citric Acid | 0.00-0.10 |
| Trisodium Ethylenediamine Disuccinate | 0.00-0.10 |
| Methylisothiazolinone and Water | 0.00-0.10 |
| Fragrance | 0.30-1.0 |
| Sodium Hydroxide | 0.00-0.30 |

### Example 22

### Shampoo Composition

An ultra-moisturizing shampoo composition can be prepared by combining the ingredients listed in Table 22 such that the composition contains the indicated ingredients in the disclosed ranges (in percentages by weight). In some cases, the Silicone Quaternium-22 of the composition shown in Table 22 can be partially or fully replaced with 0.30-1.0 weight percent of Silicone Quaternium-12.

TABLE 22
Example of an ultra moisturizing shampoo composition.

| | % in |
|---|---|
| Ingredient INCI Name | Formulation |
| Water | Balance |
| Cocamidopropyl Betaine | 10.00-30.00 |
| Sodium Methyl 2-Sulfolaurate, Disodium 2-Sulfolaurate, | 10.00-30.00 |
| and Sodium Lauryl Sulfoacetate | |
| Sodium Cocoyl Isethionate | 3.00-10.00 |
| Divinyldimethicone/Dimethicone Copolymer, C12-13 | 1.00-3.00 |
| Pareth-23, and C12-13 Pareth-3 | |
| Glycol Distearate | 0.30-1.00 |
| Silicone Quaternium-22 | 0.10-0.30 |
| Guar Hydroxypropyltrimonium Chloride | 0.30-1.00 |
| Potassium Sorbate | 0.10-0.30 |
| Citric Acid | 0.10-0.30 |
| Trisodium Ethylenediamine Disuccinate | 0.00-0.10 |
| Methylisothiazolinone and Water | 0.00-0.10 |
| Fragrance | 0.30-1.00 |
| Sodium Hydroxide | 0.00-0.30 |

### Example 23

### Shampoo Composition

A volumizing shampoo composition can be prepared by combining the ingredients listed in Table 23 such that the composition contains the indicated ingredients in the disclosed ranges (in percentages by weight). In some cases, the Silicone Quaternium-22 of the composition shown in Table 23 can be partially or fully replaced with 0.30-1.0 weight percent of Silicone Quaternium-12.

TABLE 23
Example of a volumizing shampoo composition.

| Ingredient INCI Name | % in Formulation |
|---|---|
| Water | Balance |
| Cocamidopropyl Betaine | 10.00-30.00 |
| Sodium Methyl 2-Sulfolaurate, | 10.00-30.00 |
| Disodium 2-Sulfolaurate, and | |
| Sodium lauryl Sulfoacetate | |
| Sodium Cocoyl Isethionate | 3.00-10.00 |
| Silicone Quaternium-22 | 0.10-0.30 |
| Guar Hydroxypropyltrimonium | 0.10-0.30 |
| Chloride | |
| Potassium Sorbate | 0.10-0.30 |
| Citric Acid | 0.00-0.10 |
| Trisodium Ethylenediamine | 0.00-0.10 |
| Disuccinate | |
| Methylisothiazolinone and Water | 0.00-0.10 |
| Fragrance | 0.30-1.00 |
| Sodium Hydroxide | 0.00-0.30 |

### Example 24

### Shampoo Composition

A detoxifying shampoo composition can be prepared by combining the ingredients listed in Table 24 such that the composition contains the indicated ingredients in the disclosed ranges (in percentages by weight). In some cases, the Silicone Quaternium-22 of the composition shown in Table 24 can be partially or fully replaced with 0.30-1.0 weight percent of Silicone Quaternium-12.

TABLE 24
Example of a luxury detoxifying shampoo composition.

| | % in |
|---|---|
| Ingredient INCI Name | Formulation |
| Water | Balance |
| Cocamidopropyl Betaine | 10.00-30.00 |
| Sodium Methyl 2-Sulfolaurate, Disodium 2-Sulfolaurate, | 10.00-30.00 |
| and Sodium Lauryl Sulfoacetate | |
| Sodium Cocoyl Isethionate | 3.00-10.00 |
| Polystyrene Sulfonate | 0.30-1.00 |
| Vinegar | 0.30-1.00 |
| Panthenol | 0.30-1.00 |
| Silicone Quaternium-22 | 0.30-1.00 |
| Guar Hydroxypropyltrimonium Chloride | 0.10-0.30 |
| Potassium Sorbate | 0.10-0.30 |
| Helianthus Annuus (Sunflower) Seed Extract and | 0.10-0.30 |
| Butylene Glycol | |
| Urtica Diocica (Nettle) Leaf Extract | 0.10-0.30 |
| Rosmarinus Officinalis (Rosemary) Leaf Extract | 0.10-0.30 |
| Hydrolyzed Soy Protein | 0.10-0.30 |
| Santalum Album (Sandalwood) Extract, Phellodendron | 0.00-0.10 |
| Amurense Bark Extract, and Hordeum Distichon (Barley) | |
| Extract | |
| Citric Acid | 0.00-0.10 |
| Creatine | 0.00-0.10 |
| Trisodium Ethylenediamine Disuccinate | 0.00-0.10 |
| Methylisothiazolinone and Water | 0.00-0.10 |
| Niacinamide, Sodium Starch Octenylsuccinate, Calcium | 0.00-0.10 |
| Pantothenate, Maltodextrin, Sodium Ascorbyl Phosphate, | |
| Tocopheryl Acetate, Pyridoxine HCl, and Silica | |
| Fragrance | 0.30-1.00 |
| Sodium Hydroxide | 0.00-0.30 |

### Example 25

### Shampoo Composition

A luxury moisture shampoo composition can be prepared by combining the ingredients listed in Table 25 such that the composition contains the indicated ingredients in the disclosed ranges (in percentages by weight). In some cases, the Silicone Quaternium-22 of the composition shown in Table 23 can be partially or fully replaced with 0.30-1.0 weight percent of Silicone Quaternium-12.

TABLE 25
Example of a luxury moisture shampoo.

| | % in |
|---|---|
| Ingredient INCI Name | Formulation |
| Water | Balance |
| Cocamidopropyl Betaine | 10.00-30.00 |
| Sodium Methyl 2-Sulfolaurate, Disodium 2-Sulfolaurate, | 10.00-30.00 |
| and Sodium Lauryl Sulfoacetate | |
| Sodium Cocoyl Isethionate | 3.00-10.00 |
| Fragrance | 0.30-1.00 |
| Divinyldimethicone/Dimethicone Copolymer, C12-13 | 0.30-1.00 |
| Pareth-23, and C12-13 Pareth-3 | |
| Glycol Distearate | 0.30-1.00 |
| Panthenol | 0.30-1.00 |
| Silicone Quaternium-22 | 0.30-1.00 |
| Guar Hydroxypropyltrimonium Chloride | 0.30-1.00 |
| Potassium Sorbate | 0.10-0.30 |
| Helianthus Annuus (Sunflower) Seed Extract and | 0.10-0.30 |
| Butylene Glycol | |
| Cocos Nucifera (Coconut) Oil | 0.00-0.10 |
| Persea Gratisssima (Avocado) Oil | 0.00-0.10 |
| Simmondsia Chinensis (Jojoba) Seed Oil | 0.00-0.10 |
| Hydrolyzed Soy Protein | 0.00-0.10 |
| Santalum Album (Sandalwood) Extract, Phellodendron | 0.00-0.10 |
| Amurense Bark Extract, and Hordeum Distichon (Barley) | |
| Extract | |
| Citric Acid | 0.00-0.10 |
| Creatine | 0.00-0.10 |
| Trisodium Ethylenediamine Disuccinate | 0.00-0.10 |
| Methylisothiazolinone and Water | 0.00-0.10 |
| Niacinamide, Sodium Starch Octenylsuccinate, Calcium | 0.00-0.10 |
| Pantothenate, Maltodextrin, Sodium Ascorbyl Phosphate, | |
| Tocopheryl Acetate, Pyridoxine HCl, and Silica | |
| Fragrance | 0.00-0.30 |
| Sodium Hydroxide | 10.00-30.00 |

### Example 26

### Shampoo Composition

A luxury ultra moisture shampoo composition can be prepared by combining the ingredients listed in Table 26 such that the composition contains the indicated ingredients in the disclosed ranges (in percentages by weight). In some cases, the Silicone Quaternium-22 of the composition shown in Table 26 can be partially or fully replaced with 0.30-1.0 weight percent of Silicone Quaternium-12.

TABLE 26
Example of a luxury ultra moisture shampoo

| | % in |
|---|---|
| Ingredient INCI Name | Formulation |
| Water | Balance |
| Cocamidopropyl Betaine | 10.00-30.00 |
| Sodium Methyl 2-Sulfolaurate, Disodium 2-Sulfolaurate, | 10.00-30.00 |
| and Sodium Lauryl Sulfoacetate | |
| Sodium Cocoyl Isethionate | 3.00-10.00 |
| Divinyldimethicone/Dimethicone Copolymer, C12-13 | 1.00-3.00 |
| Pareth-23, and C12-13 Pareth-3 | |
| Glycol Distearate | 0.30-1.00 |
| Panthenol | 0.30-1.00 |
| Silicone Quaternium-22 | 0.30-1.00 |
| Guar Hydroxypropyltrimonium Chloride | 0.30-1.00 |
| Potassium Sorbate | 0.10-0.30 |
| Helianthus Annuus (Sunflower) Seed Extract and Butylene | 0.10-0.30 |
| Glycol | |
| Cocos Nucifera (Coconut) Oil | 0.00-0.10 |
| Persea Gratisssima (Avocado) Oil | 0.00-0.10 |
| Simmondsia Chinensis (Jojoba) Seed Oil | 0.00-0.10 |
| Hydrolyzed Soy Protein | 0.00-0.10 |
| Santalum Album (Sandalwood) Extract, Phellodendron | 0.00-0.10 |
| Amurense Bark Extract, and Hordeum Distichon (Barley) | |
| Extract | |
| Citric Acid | 0.00-0.10 |
| Creatine | 0.00-0.10 |
| Trisodium Ethylenediamine Disuccinate | 0.00-0.10 |
| Methylisothiazolinone and Water | 0.00-0.10 |
| Niacinamide, Sodium Starch Octenylsuccinate, Calcium | 0.00-0.10 |
| Pantothenate, Maltodextrin, Sodium Ascorbyl Phosphate, | |
| Tocopheryl Acetate, Pyridoxine HCl, and Silica | |
| Fragrance | 0.30-1.00 |
| Sodium Hydroxide | 0.00-0.30 |

### Example 27

### Shampoo Composition

A luxury volume shampoo composition can be prepared by combining the ingredients listed in Table 27 such that the composition contains the indicated ingredients in the disclosed ranges (in percentages by weight). In some cases, the Silicone Quaternium-22 of the composition shown in Table 27 can be partially or fully replaced with 0.30-1.0 weight percent of Silicone Quaternium-12.

TABLE 27
Example of luxury volume shampoo.

| | % in |
|---|---|
| Ingredient INCI Name | Formulation |
| Water | Balance |
| Cocamidopropyl Betaine | 10.00-30.00 |
| Sodium Methyl 2-Sulfolaurate, Disodium 2-Sulfolaurate, | 10.00-30.00 |
| and Sodium Lauryl Sulfoacetate | |
| Sodium Cocoyl Isethionate | 3.00-10.00 |
| Panthenol | 0.30-1.00 |
| Silicone Quatenrium-22 | 0.30-1.00 |
| Water and Urtica Dioica (Nettle) Extract | 0.10-0.30 |
| Water and Rosmarinus Officinalis (Rosemary) Leaf Extract | 0.10-0.30 |
| Guar Hydroxypropyltrimonium Chloride | 0.10-0.30 |
| Potassium Sorbate | 0.10-0.30 |
| Helianthus Annuus (Sunflower) Seed Extract and Butylene | 0.10-0.30 |
| Glycol | |
| Simmondsia Chinensis (Jojoba) Seed Oil | 0.00-0.10 |
| Hydrolyzed Soy Protein | 0.00-0.10 |
| Santalum Album (Sandalwood) Extract, Phellodendron | 0.00-0.10 |
| Amurense Bark Extract, and Hordeum Distichon (Barley) | |
| Extract | |
| Citric Acid | 0.00-0.10 |
| Creatine | 0.00-0.10 |
| Trisodium Ethylenediamine Disuccinate | 0.00-0.10 |
| Methylisothiazolinone and Water | 0.00-0.10 |
| Niacinamide, Sodium Starch Octenylsuccinate, Calcium | 0.00-0.10 |
| Pantothenate, Maltodextrin, Sodium Ascorbyl Phosphate, | |
| Tocopheryl Acetate, Pyridoxine HCl, and Silica | |
| Fragrance | 0.30-1.00 |
| Sodium Hydroxide | 0.00-0.30 |

### Example 28

### Conditioner Composition

A moisturizing composition can be prepared by combining the ingredients listed in Table 28 such that the composition contains the indicated ingredients in the disclosed ranges (in percentages by weight). In some cases, the Silicone Quaternium-22 of the composition shown in Table 28 can be partially or fully replaced with 0.30-1.0 weight percent of Silicone Quaternium-12.

TABLE 28
Example of a moisturize conditioner.

| Ingredient INCI Name | % in Formulation |
|---|---|
| Water | Balance |
| Quaternium-91, Cetrimonium Methosulfate, and | 1.00-3.00 |
| Cetearyl Alcohol | |
| Stearyl Alcohol | 1.00-3.00 |
| Distearoylethyl Dimonium Chloride and Cetearyl | 1.00-3.00 |
| Alcohol | |
| Cetyl Alcohol | 1.00-3.00 |
| Hydrogenated Polydecene | 1.00-3.00 |
| Cetearyl Alcohol | 0.300-1.00 |
| Glycerin | 1.00-3.00 |
| Propanediol | 1.00-3.00 |
| Dicaprylyl Carbonate | 1.00-3.00 |
| Fragrance | 0.30-1.00 |
| Quaternium-80 | 0.10-0.30 |
| Silicone Quaternium-22 | 0.10-0.30 |
| Stearamidopropyl Dimethylamine | 0.30-1.0 |
| Sorbic Acid | 0.10-0.30 |
| Guar Hydroxypropyltrimonium Chloride | 0.10-0.30 |
| Tetrasodium EDTA | 0.00-0.10 |
| Methylisothiazolinone and Water | 0.00-0.10 |
| Citric Acid | 0.00-0.10 |

### Example 29

### Conditioner Composition

An ultra-moisturizing composition can be prepared by combining the ingredients listed in Table 29 such that the composition contains the indicated ingredients in the disclosed ranges (in percentages by weight). In some cases, the Silicone Quaternium-22 of the composition shown in Table 29 can be partially or fully replaced with 0.30-1.0 weight percent of Silicone Quaternium-12.

TABLE 29
Example of ultra-moisturizing conditioner.

| Ingredient INCI Name | % in Formulation |
|---|---|
| Water | 80.970 |
| Brassicamidopropyl Dimethylamine | 3.00-10.00 |
| Stearyl Alcohol | 3.00-10.00 |
| Distearoylethyl Dimonium Chloride and Cetearyl | 1.00-3.00 |
| Alcohol | |
| Cetyl Alcohol | 1.00-3.00 |
| Hydrogenated Polydecene | 1.00-3.00 |
| Cetearyl Alcohol | 0.300-1.00 |
| Cetrimonium Chloride | 1.00-3.00 |
| Cyclopentasiloxane and Dimethicone Crosspolymer | 0.30-1.00 |
| Phenoxyethanol | 0.30-1.00 |
| Fragrance | 0.30-1.00 |
| Quaternium-80 | 0.10-0.30 |
| Silicone Quaternium-22 | 0.10-0.30 |
| Cyclopentasiloxane | 0.30-1.00 |
| Lactic Acid | 0.30-1.00 |
| Sorbic Acid | 0.10-0.30 |
| Guar Hydroxypropyltrimonium Chloride | 0.10-0.30 |
| Tetrasodium EDTA | 0.10-0.30 |
| Citric Acid | 0.30-1.0 |

### Example 30

### Conditioner Composition

A volumizing composition can be prepared by combining the ingredients listed in Table 30 such that the composition contains the indicated ingredients in the disclosed ranges (in percentages by weight). In some cases, the Silicone Quaternium-22 of the composition shown in Table 30 can be partially or fully replaced with 0.30-1.0 weight percent of Silicone Quaternium-12.

TABLE 30
Example of volumizing conditioner.

| Ingredient INCI Name | % in Formulation |
|---|---|
| Water | Balance |
| Behentrimonium Methosulfate and Cetearyl Alcohol | 1.00-3.00 |
| Stearyl Alcohol | 1.00-3.00 |
| Distearoylethyl Dimonium Chloride and Cetearyl | 1.00-3.00 |
| Alcohol | |
| Cetyl Alcohol | 0.30-1.00 |
| Hydrogenated Polydecene | 0.30-1.00 |
| Cetearyl Alcohol | 0.30-1.00 |
| Propanediol | 1.00-3.00 |
| PPG-3 Benzyl Ether Ethylhexanoate | 0.30-1.00 |
| Dicaprylyl Carbonate | 0.10-0.30 |
| Fragrance | 0.30-1.00 |
| Silicone Quaternium-22 | 0.10-0.30 |
| Stearamidopropyl Dimethylamine | 0.30-1.00 |
| Sorbic Acid | 0.10-0.30 |
| Guar Hydroxypropyltrimonium Chloride | 0.10-0.30 |
| Tetrasodium EDTA | 0.00-0.10 |
| Methylisothiazolinone and Water | 0.00-0.10 |

### Example 31

### Conditioner Composition

A luxury moisturize crème rinse composition can be prepared by combining the ingredients listed in Table 31 such that the composition contains the indicated ingredients in the disclosed ranges (in percentages by weight). In some cases, the Silicone Quaternium-22 of the composition shown in Table 31 can be partially or fully replaced with 0.30-1.0 weight percent of Silicone Quaternium-12.

TABLE 31
Example of luxury moisture crème rinse.

| | % in |
|---|---|
| Ingredient INCI Name | Formulation |
| Water | Balance |
| Quaternium-91, Cetrimonium Methosulfate, and Cetearyl | 1.00-3.00 |
| Alcohol | |
| Stearyl Alcohol | 1.00-3.00 |
| Distearoylethyl Dimonium Chloride and Cetearyl Alcohol | 1.00-3.00 |
| Cetyl Alcohol | 1.00-3.00 |
| Hydrogenated Polydecene | 1.00-3.00 |
| Cetearyl Alcohol | 1.00-3.00 |
| Glycerin | 1.00-3.00 |
| Propanediol | 1.00-3.00 |
| PPG-3 Benzyl Ether Ethylhexanoate | 1.00-3.00 |
| Dicaprylyl Carbonate | 1.00-3.00 |
| Fragrance | 0.30-1.00 |
| Quaternium-80 | 0.30-1.00 |
| Silicone Quaternium-22 | 0.30-1.00 |
| Stearamidopropyl Dimethylamine | 0.30-1.00 |
| Panthenol | 0.30-1.00 |
| Santalum Album (Sandalwood) Extract, Phellodendron | 0.30-1.00 |
| Amurense Bark Extract, and Hordeum Distichon (Barley) | |
| Extract | |
| Hydrolyzed Soy Protein | 0.10-0.30 |
| Helianthus Annuus (Sunflower) Seed Extract and Butylene | 0.10-0.30 |
| Glycol | |
| Sorbic Acid | 0.10-0.30 |
| Guar Hydroxypropyltrimonium Chloride | 0.10-0.30 |
| Cocos Nucifera (Coconut) Oil | 0.00-0.10 |
| Persea Gratisssima (Avocado) Oil | 0.00-0.10 |
| Simmondsia Chinensis (Jojoba) Seed Oil | 0.00-0.10 |
| Phytantriol | 0.00-0.10 |
| Creatine | 0.00-0.10 |
| Tetrasodium EDTA | 0.00-0.10 |
| Methylisothiazolinone and Water | 0.00-0.10 |
| Niacinamide, Sodium Starch Octenylsuccinate, Calcium | 0.00-0.10 |
| Pantothenate, Maltodextrin, Sodium Ascorbyl Phosphate, | |
| Tocopheryl Acetate, Pyridoxine HCl, and Silica | |
| Citric Acid | 0.00-0.100 |

### Example 32

### Conditioner Composition

A luxury ultra moisturize crème rinse composition can be prepared by combining the ingredients listed in Table 32 such that the composition contains the indicated ingredients in the disclosed ranges (in percentages by weight). In some cases, the Silicone Quaternium-22 of the composition shown in Table 32 can be partially or fully replaced with 0.30-1.0 weight percent of Silicone Quaternium-12.

TABLE 32
Example of luxury ultra moisture crème rinse.

| | % in |
|---|---|
| Ingredient INCI Name | Formulation |
| Water | Balance |
| Brassicamidopropyl Dimethylamine | 3.00-10.00 |
| Stearyl Alcohol | 3.00-10.00 |
| Distearoylethyl Dimonium Chloride and Cetearyl Alcohol | 1.00-3.00 |
| Cetyl Alcohol | 1.00-3.00 |
| Hydrogenated Polydecene | 1.00-3.00 |
| Cetearyl Alcohol | 1.00-3.00 |
| Cetrimonium Chloride | 1.00-3.00 |
| Cyclopentasiloxane and Dimethicone Crosspolymer | 0.30-1.00 |
| Phenoxyethanol | 0.30-1.00 |
| Fragrance | 0.30-1.00 |
| Quaternium-80 | 0.30-1.00 |
| Silicone Quaternium-22 | 0.30-1.00 |
| Stearamidopropyl Dimethylamine | 0.30-1.00 |
| Panthenol | 0.30-1.00 |
| Santalum Album (Sandalwood) Extract, Phellodendron | 0.30-1.00 |
| Amurense Bark Extract, and Hordeum Distichon (Barley) | |
| Extract | |
| Cyclopentasiloxane | 0.30-1.00 |
| Hydrolyzed Soy Protein | 0.10-0.30 |
| Helianthus Annuus (Sunflower) Seed Extract and Butylene | 0.10-0.30 |
| Glycol | |
| Lactic Acid | 0.30-1.00 |
| Sorbic Acid | 0.10-0.30 |
| Citric Acid | 0.30-1.00 |
| Guar Hydroxypropyltrimonium Chloride | 0.10-0.30 |
| Cocos Nucifera (Coconut) Oil | 0.30-1.00 |
| Persea Gratisssima (Avocado) Oil | 0.30-1.00 |
| Simmondsia Chinensis (Jojoba) Seed Oil | 0.30-1.00 |
| Phytantriol | 0.00-0.10 |
| Creatine | 0.00-0.10 |
| Tetrasodium EDTA | 0.10-0.30 |

### Example 33

### Conditioner Composition

A luxury volumizing crème rinse composition can be prepared by combining the ingredients listed in Table 33 such that the composition contains the indicated ingredients in the disclosed ranges (in percentages by weight). In some cases, the Silicone Quaternium-22 of the composition shown in Table 33 can be partially or fully replaced with 0.30-1.0 weight percent of Silicone Quaternium-12.

TABLE 33
Example of luxury volume crème rinse.

| Ingredient INCI Name | % in Formulation |
|---|---|
| Water | Balance |
| Behentrimonium Methosulfate and Cetearyl Alcohol | 1.00-3.00 |
| Stearyl Alcohol | 1.00-3.00 |
| Distearoylethyl Dimonium Chloride and Cetearyl | 1.00-3.00 |
| Alcohol | |
| Cetyl Alcohol | 0.30-1.00 |
| Hydrogenated Polydecene | 0.30-1.00 |
| Cetearyl Alcohol | 1.00-3.00 |
| Propanediol | 1.00-3.00 |
| PPG-3 Benzyl Ether Ethylhexanoate | 0.30-1.00 |
| Dicaprylyl Carbonate | 0.10-0.30 |
| Fragrance | 0.30-1.00 |
| Quaternium-80 | 0.30-1.00 |
| Silicone Quaternium-22 | 0.30-1.00 |
| Stearamidopropyl Dimethylamine | 0.30-1.00 |
| Panthenol | 0.10-0.30 |
| Santalum Album (Sandalwood) Extract, Phellodendron | 0.30-1.00 |
| Amurense Bark Extract, and Hordeum Distichon | |
| (Barley) Extract | |
| Hydrolyzed Soy Protein | 0.10-0.30 |
| Helianthus Annuus (Sunflower) Seed Extract and | 0.10-0.30 |
| Butylene Glycol | |
| Sorbic Acid | 0.10-0.30 |
| Guar Hydroxypropyltrimonium Chloride | 0.10-0.30 |
| Water and Urtica Dioica (Nettle) Extract | 0.00-0.10 |
| Water and Rosmarinus Officinalis (Rosemary) Extract | 0.00-0.10 |
| Phytantriol | 0.00-0.10 |
| Creatine | 0.00-0.10 |
| Tetrasodium EDTA | 0.00-0.10 |
| Methylisothiazolinone and Water | 0.00-0.10 |
| Niacinamide, Sodium Starch Octenylsuccinate, | 0.00-0.10 |
| Calcium Pantothenate, Maltodextrin, Sodium | |
| Ascorbyl Phosphate, Tocopheryl Acetate, | |
| Pyridoxine HCl, and Silica | |

### Example 34

### Repair Hair Masque Composition

A luxury repair hair masque composition can be prepared by combining the ingredients listed in Table 34 such that the composition contains the indicated ingredients in the disclosed ranges (in percentages by weight). In some cases, the Silicone Quaternium-22 of the composition shown in Table 34 can be partially or fully replaced with 1.00-3.00 weight percent of Silicone Quaternium-12.

TABLE 34
Example of luxury repair hair masque.

| | % in |
|---|---|
| Ingredient INCI Name | Formulation |
| Water | Balance |
| Brassicamidopropyl Dimethylamine | 3.000-10.00 |
| Stearyl Alcohol | 3.00-10.00 |
| Distearoylethyl Dimonium Chloride and Cetearyl Alcohol | 0.10-0.30 |
| Cetyl Alcohol | 3.00-10.00 |
| Hydrogenated Polydecene | 1.00-3.00 |
| Quaternium-91, Cetrimonium Methosulfate, and Cetearyl | 1.00-3.00 |
| Alcohol | |
| Glycerin | 3.00-10.00 |
| Dimethicone | 1.00-3.00 |
| Cyclopentasiloxane | 1.00-3.00 |
| Butyrospermum Parkii (Shea Butter) | 1.00-3.00 |
| Cetearyl Alcohol | 1.00-3.00 |
| PPG-3 Benzyl Ether Ethylhexanoate | 1.00-3.00 |
| Amodimethicone, Trideceth-12, and Cetrimonium Chloride | 0.30-1.00 |
| Cyclopentasiloxane and Dimethicone Crosspolymer | 0.30-1.00 |
| Fragrance | 0.30-1.00 |
| Quaternium-80 | 0.30-1.00 |
| Silicone Quaternium-22 | 1.00-3.00 |
| Stearamidopropyl Dimethylamine | 0.30-1.00 |
| Panthenol | 1.00-3.00 |
| Santalum Album (Sandalwood) Extract, Phellodendron | 1.00-3.00 |
| Amurense Bark Extract, and Hordeum Distichon (Barley) | |
| Extract | |
| Hydrolyzed Soy Protein | 0.10-0.30 |
| Helianthus Annuus (Sunflower) Seed Extract and Butylene | 0.10-0.30 |
| Glycol | |
| Lactic Acid | 0.30-1.00 |
| Sorbic Acid | 0.10-0.30 |
| Guar Hydroxypropyltrimonium Chloride | 0.10-0.30 |
| Cocos Nucifera (Coconut) Oil | 0.10-0.30 |
| Persea Gratisssima (Avocado) Oil | 0.10-0.30 |
| Simmondsia Chinensis (Jojoba) Seed Oil | 0.10-0.30 |
| Phytantriol | 0.00-0.10 |
| Creatine | 0.00-0.10 |
| Tetrasodium EDTA | 0.10-0.30 |
| Methylisothiazolinone and Water | 0.00-0.10 |
| Niacinamide, Sodium Starch Octenylsuccinate, Calcium | 0.00-0.10 |
| Pantothenate, Maltodextrin, Sodium Ascorbyl Phosphate, | |
| Tocopheryl Acetate, Pyridoxine HCl, and Silica | |
| Citric Acid | 0.30-1.00 |

### Example 35

### Leave-In Detangling Spray Composition

A leave-in detangling spray composition can be prepared by combining the ingredients listed in Table 35 such that the composition contains the indicated ingredients in the disclosed ranges (in percentages by weight). In some cases, the Silicone Quaternium-22 of the composition shown in Table 35 can be partially or fully replaced with 0.30-1.0 weight percent of Silicone Quaternium-12.

TABLE 35
Example of a leave-in detangling spray composition.

| Ingredient INCI Name | % in Formulation |
|---|---|
| Water | Balance |
| Cetrimonium Chloride | 1.00-3.00 |
| Bis(C13-15 Alkoxy) PG-Amodimethicone | 1.00-3.00 |
| Caprylyl Glycol and 1,2-Hexandediol | 0.30-1.00 |
| Phenoxyethanol | 0.30-1.00 |
| Fragrance | 0.30-1.00 |
| Panthenol | 0.10-0.30 |
| Silicone Quaternium-22 | 0.30-1.00 |
| Polysorbate 80 | 0.10-0.30 |
| Helianthus Annuus (Sunflower) Seed Extract and | 0.00-0.10 |
| Butylene Glycol | |
| Hydrolyzed Soy Protein | 0.00-0.10 |
| Santalum Album (Sandalwood) Extract, Phellodendron | 0.00-0.10 |
| Amurense Bark Extract, and Hordeum Distichon | |
| (Barley) Extract | |
| Creatine | 0.00-0.10 |
| Aminomethyl Propanol | 0.00-0.10 |
| Disodium EDTA | 0.10-0.30 |
| Niacinamide, Sodium Starch Octenylsuccinate, | 0.00-0.100 |
| Calcium Pantothenate, Maltodextrin, Sodium | |
| Ascorbyl Phosphate, Tocopheryl Acetate, | |
| Pyridoxine HCl, and Silica | |

### Example 36

### Heat Protection Spray Composition

A heat protection spray composition can be prepared by combining the ingredients listed in Table 36 such that the composition contains the indicated ingredients in the disclosed ranges (in percentages by weight). In some cases, the Silicone Quaternium-22 of the composition shown in Table 36 can be partially or fully replaced with 0.00-0.10 weight percent of Silicone Quaternium-12.

TABLE 36
Example of luxury hot iron prep spray.

| | % in |
|---|---|
| Ingredient INCI Name | Formulation |
| Water | Balance |
| Cetrimonium Chloride | 1.00-3.00 |
| Glycerin | 1.00-3.00 |
| Sodium Cocoyl Amino Acids, Potassium Dimethicone | 1.00-3.00 |
| PEG-7 Panthenyl Phosphate | |
| Sodium Laneth-40 Maleate/Styrene Sulfonate Copolymer | 1.00-3.00 |
| Polysorbate 20 | 0.30-1.00 |
| Phenoxyethanol | 0.30-1.00 |
| PEG-12 Dimethicone | 0.30-1.00 |
| Panthenol | 0.30-1.00 |
| Fragrance | 0.30-1.00 |
| Phytantriol | 0.00-0.10 |
| Silicone Quaternium-22 | 0.00-0.10 |
| Potassium Sorbate | 0.10-0.30 |
| Aminomethyl Propanol | 0.00-0.10 |
| Helianthus Annuus (Sunflower) Seed Extract and Butylene | 0.00-0.10 |
| Glycol | |
| Hydrolyzed Soy Protein | 0.10-0.30 |
| Santalum Album (Sandalwood) Extract, Phellodendron | 0.00-0.10 |
| Amurense Bark Extract, and Hordeum Distichon (Barley) | |
| Extract | |
| Creatine | 0.00-0.10 |
| Disodium EDTA | 0.10-0.30 |
| Niacinamide, Sodium Starch Octenylsuccinate, Calcium | 0.00-0.10 |
| Pantothenate, Maltodextrin, Sodium Ascorbyl Phosphate, | |
| Tocopheryl Acetate, Pyridoxine HCl, and Silica | |

### Example 37

### Non-Aerosol Hair Spray Composition

A non-aerosol hair spray composition can be prepared by combining the ingredients listed in Table 37 such that the composition contains the indicated ingredients in the disclosed ranges (in percentages by weight). In some cases, the Silicone Quaternium-22 of the composition shown in Table 37 can be partially or fully replaced with 0.30-1.0 weight percent of Silicone Quaternium-12.

TABLE 37
Example of luxury classic hold finishing spray.

| Ingredient INCI Name | % in Formulation |
|---|---|
| SD Alcohol 40-B | Balance |
| Water | 30.00-50.00 |
| Acrylates/Hydroxyesters Acrylates Copolymer | 3.00-10.00 |
| VA/Crotonates/Vinyl Neodecanoate Copolymer | 1.00-3.00 |
| Aminomethyl Propanol | 0.30-1.00 |
| Fragrance | 0.30-1.00 |
| Polyquaternium-69 | 0.10-0.30 |
| Lauryl Pyrrolidone | 0.10-0.30 |
| Panthenol | 0.10-0.30 |
| Silicone Quaternium-22 | 0.00-0.10 |
| PEG-12 Dimethicone | 0.10-0.30 |
| Helianthus Annuus (Sunflower) Seed Extract and | 0.00-0.10 |
| Butylene Glycol | |
| Water, Butylene Glycol, and Vitis Vinifera (Grape) | 0.00-0.10 |
| Leaf Extract | |
| Water (and) Urtica Dioica (Nettle) Extract | 0.00-0.10 |
| Rosmarinus Officinalis (Rosemary) Leaf Extract | 0.00-0.10 |
| Hydrolyzed Soy Protein | 0.00-0.10 |
| Santalum Album (Sandalwood) Extract, Phellodendron | 0.00-0.10 |
| Amurense Bark Extract, and Hordeum Distichon | |
| (Barley) Extract | |
| Creatine | 0.00-0.10 |
| Phytantriol | 0.00-0.10 |
| Niacinamide, Sodium Starch Octenylsuccinate, | 0.00-0.10 |
| Calcium Pantothenate, Maltodextrin, Sodium | |
| Ascorbyl Phosphate, Tocopheryl Acetate, | |
| Pyridoxine HCl, and Silica | |

### Example 38

### Styling Gel Composition

A styling gel composition can be prepared by combining the ingredients listed in Table 38 such that the composition contains the indicated ingredients in the disclosed ranges (in percentages by weight). In some cases, the Silicone Quaternium-22 of the composition shown in Table 38 can be partially or fully replaced with 0.10-0.30 weight percent of Silicone Quaternium-12.

TABLE 38
Example of luxury defining gel.

| Ingredient INCI Name | % in Formulation |
|---|---|
| Water | Balance |
| AMP-Acrylates/Alkyl Methacrylate Copolymer | 3.00-10.00 |
| Polyacrylamide-2 Crosspolymer | 3.00-10.00 |
| Aminomethyl Propanol | 1.00-3.00 |
| Acrylates/C10-30 Alkyl Acrylate Crosspolymer | 0.30-1.00 |
| PVP | 0.30-1.00 |
| Polyquaternium-69 | 0.30-1.00 |
| PEG-40 Hydrogenated Castor Oil | 0.30-1.00 |
| Phenoxyethanol | 0.30-1.00 |
| Panthenol | 0.30-1.00 |
| Glycerin and Glyceryl Acrylates/Acrylic Acid | 0.30-1.00 |
| Copolymer | |
| Glycerin | 0.10-0.30 |
| Fragrance | 0.10-0.30 |
| Tetrasodium EDTA | 0.10-0.30 |
| PEG-12 Dimethicone | 0.10-0.30 |
| Silicone Quaternium-22 | 0.10-0.30 |
| Phytantriol | 0.00-0.10 |
| Helianthus Annuus (Sunflower) Seed Extract and | 0.00-0.10 |
| Butylene Glycol | |
| Hydrolyzed Soy Protein | 0.00-0.10 |
| Santalum Album (Sandalwood) Extract, Phellodendron | 0.00-0.10 |
| Amurense Bark Extract, and Hordeum Distichon | |
| (Barley) Extract | |
| Creatine | 0.00-0.10 |
| Methylisothiazolinone and Water | 0.00-0.10 |
| Niacinamide, Sodium Starch Octenylsuccinate, | 0.00-0.10 |
| Calcium Pantothenate, Maltodextrin, Sodium | |
| Ascorbyl Phosphate, Tocopheryl Acetate, | |
| Pyridoxine HCl, and Silica | |

### Example 39

### Styling Crème Composition

A styling crème composition can be prepared by combining the ingredients listed in Table 39 such that the composition contains the indicated ingredients in the disclosed ranges (in percentages by weight). In some cases, the Silicone Quaternium-22 of the composition shown in Table 39 can be partially or fully replaced with 0.30-1.0 weight percent of Silicone Quaternium-12.

TABLE 39
Example of luxury soft hold styling crème.

| Ingredient INCI Name | % in Formulation |
|---|---|
| Water | Balance |
| Sorbitol | 1.00-3.00 |
| Hydrogenated Polydecene | 1.00-3.00 |
| Dehydroxanthan Gum | 1.00-3.00 |
| Phenoxyethanol | 0.30-1.00 |
| Polyquaternium-69 | 0.30-1.00 |
| PPG-3 Benzyl Ether Ethylhexanoate | 0.30-1.00 |
| Fragrance | 0.300-1.00 |
| Silicone Quaternium-22 | 0.30-1.00 |
| Polyquaternium-4/Hydroxypropyl Starch Copolymer | 0.10-0.30 |
| Sodium Polystyrene Sulfonate | 0.10-0.30 |
| Panthenol | 0.30-1.00 |
| Santalum Album (Sandalwood) Extract, | 0.30-1.00 |
| Phellodendron Amurense Bark Extract, and | |
| Hordeum Distichon (Barley) Extract | |
| Hydrolyzed Soy Protein | 0.10-0.30 |
| Helianthus Annuus (Sunflower) Seed Extract and | 0.00-0.10 |
| Butylene Glycol | |
| Persea Gratisssima (Avocado) Oil | 0.00-0.10 |
| Simmondsia Chinensis (Jojoba) Seed Oil | 0.00-0.10 |
| Phytantriol | 0.00-0.10 |
| Creatine | 0.00-0.10 |
| Trisodium Ethylenediamine Disuccinate | 0.00-0.10 |
| Methylisothiazolinone and Water | 0.00-0.10 |
| Niacinamide, Sodium Starch Octenylsuccinate, | 0.00-0.10 |
| Calcium Pantothenate, Maltodextrin, Sodium | |
| Ascorbyl Phosphate, Tocopheryl Acetate, | |
| Pyridoxine HCl, and Silica | |
| Citric Acid | 0.00-0.10 |

### Example 40

### Luxury Texturizing Pomade Composition

A luxury texturizing pomade composition can be prepared by combining the ingredients listed in Table 40 such that the composition contains the indicated ingredients in the disclosed ranges (in percentages by weight). In some cases, the Silicone Quaternium-22 of the composition shown in Table 40 can be partially or fully replaced with 0.30-1.0 weight percent of Silicone Quaternium-12.

TABLE 20
Luxury Texturizing Pomade

| Ingredient INCI Name | % in Formulation |
|---|---|
| Water | Balance |
| Isoceteth-20 | 10.00-30.00 |
| PPG-3 Benzyl Ether Ethylhexanoate | 10.00-30.00 |
| Oleth-2 | 3.00-10.00 |
| Propanediol | 3.00-10.00 |
| Polyurethane-14 and AMP-Acrylates Copolymer | 3.00-10.00 |
| Sorbitol | 1.00-3.00 |
| Sodium Polystyrene Sulfonate | 1.00-3.00 |
| PVP | 1.00-3.00 |
| Fragrance | 0.30-1.00 |
| Panthenol | 0.10-0.30 |
| Silicone Quaternium-22 | 0.30-1.00 |
| Polyquaternium-4/Hydroxypropyl Starch Copolymer | 0.10-0.30 |
| Helianthus Annuus (Sunflower) Seed Extract and | 0.10-0.30 |
| Butylene Glycol | |
| Cocos Nucifera (Coconut) Oil | 0.00-0.10 |
| Persea Gratisssima (Avocado) Oil | 0.00-0.10 |
| Simmondsia Chinensis (Jojoba) Seed Oil | 0.00-0.10 |
| Hydrolyzed Soy Protein | 0.10-0.30 |
| Santalum Album (Sandalwood) Extract, Phellodendron | 0.30-1.00 |
| Amurense Bark Extract, and Hordeum Distichon | |
| (Barley) Extract | |
| Phytantriol | 0.00-0.10 |
| Creatine | 0.00-0.10 |
| Tetrasodium EDTA | 0.00-0.10 |
| Methylisothiazolinone and Water | 0.00-0.10 |
| Niacinamide, Sodium Starch Octenylsuccinate, | 0.00-0.10 |
| Calcium Pantothenate, Maltodextrin, Sodium | |
| Ascorbyl Phosphate, Tocopheryl Acetate, | |
| Pyridoxine HCl, and Silica | |

### Example 41

### Analysis of Hair Care Compositions

Three shampoo compositions (i.e., the shampoo compositions described in Examples 5, 6, and 7) were produced and tested for the ability to be used with minimal fading of a previously applied hair color. In particular, double bleached platinum blonde test hair tresses (4 grams in weight and 7 inches in length) were dyed with a commercial permanent red hair dye and then measured using reflectance spectrometry to establish a starting red hair value. The hair switches were grouped into sets of three and then treated with the shampoo compositions (0.50 grams of shampoo followed by 30 seconds of lathering and 2 minutes of rinsing with clean water for each shampoo cycle).

A competitor shampoo composition (i.e., the Pureology Hydrate Shampoo®) was also tested for comparison purposes as it is proclaimed to clean hair without stripping color. After the first, fifth, and tenth shampooing cycles, the red color difference for each test tress as compared to the starting red hair value was determined using reflectance spectrometry. The results are presented in Table 41 with a number closer to zero representing better color wash fastness (e.g., less red color loss). The shampoo compositions described in Examples 5, 6, and 7 performed very well, exhibiting a color wash fastness that is at least as effective as the competitive shampoo (Table 34).

TABLE 41
Color loss after shampoo cycles.

| Sample | 1X | 5X | 10X |
|---|---|---|---|
| Competitive Shampoo | −1.56 | −3.44 | −4.29 |
| Shampoo (Example 5) | −1.37 | −3.12 | −3.92 |
| Shampoo (Example 6) | −1.09* | −2.95 | −3.76* |
| Shampoo (Example 7) | −1.34 | −3.13 | −3.86 |

*= Significantly different from competitive shampoo at 95% confidence.

These results demonstrate that the hair care compositions provided herein can be used with minimal fading of a previously applied hair color.

### Example 42

### Analysis of Hair Care Compositions

Four conditioner compositions (i.e., the conditioner compositions described in Examples 11, 12, 13, and 14) were produced and tested for the ability to provide thermal protection. In particular, bleached human hair (4 grams weight and 7 inches long) was treated with 0.50 grams of each conditioner, allowed to rest on the hair for 30 seconds and then rinsed for 2 minutes with clean water. The hair was allowed to dry at ambient temperature and then exposed to heat from a flat iron for 10 seconds followed by a 30 second rest and then heated again for a total of three complete heat cycles. Hair samples from each individual hair switch were analyzed using differential scanning calorimetry to determine the heat resistance benefit imparted to the hair by each conditioner treatment.

A competitor conditioner composition (i.e., the Pureology Hydrate Conditioner®) was also tested for comparison purposes as it is proclaimed to protect against heat damage. A conditioner formulation containing 1 percent cetyl trimethyl ammonium chloride (CTAC) was used as a control for the differential scanning calorimetry measurements.

The results are presented in Table 42 with a higher number representing better thermal protection. The conditioner compositions described in Examples 11, 12, 13, and 14 performed very well, exhibiting thermal protection that is at least equally effective as the competitive conditioner and in three of the four compositions significantly better thermal protection (Table 42).

TABLE 42
Thermal protection of selected conditioners.

| Sample | |
|---|---|
| Competitive Conditioner | 151.7 |
| Conditioner (Example 11) | 152.3* |
| Conditioner (Example 12) | 153.0* |
| Conditioner (Example 13) | 151.9 |
| Conditioner (Example 14) | 153.6* |

*= Significantly different from competitive conditioner at 95% confidence.

These results demonstrate that the hair care compositions provided herein can be used to provide thermal protection.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A hair care composition selected from the group consisting of shampoos, styling gels, aerosol styling sprays, non-aerosol styling sprays, aerosol styling mousses, styling gels, styling pomades, and thermal protection sprays, wherein said hair care composition comprises between about 0.01 percent and about 5 percent of a quaternized polysiloxane.

2. The composition of claim 1, wherein said composition is a shampoo.

3. The composition of claim 1, wherein said composition comprises between about 0.01 percent and about 1.5 percent of said quaternized polysiloxane.

4. The composition of claim 1, wherein said composition comprises between about 0.1 percent and about 1.5 percent of said quaternized polysiloxane.

5. The composition of claim 1, wherein said quaternized polysiloxane is silicone quaterium 12 or 22.

6. The composition of claim 1, wherein said composition comprises one or more zwitterionic surfactants.

7. The composition of claim 1, wherein said composition comprises cocamidopropyl betaine.

8. The composition of claim 1, wherein said composition comprises one or more anionic surfactants.

9. The composition of claim 1, wherein said composition comprises sodium cocoyl isethionate, sodium methyl 2-sulfolaurate, disodium 2-sulfolaurate, sodium lauryl sulfoacetate, or a combination thereof.

10. The composition of claim 1, wherein said composition comprises one or more botanical compounds.

11. The composition of claim 1, wherein said composition comprises a sunflower seed extract, a sandalwood extract, a Phellodendron amurense bark extract, a barley extract, or a combination thereof.

12. The composition of claim 1, wherein said composition comprises one or more amino acids.

13. The composition of claim 1, wherein said composition comprises creatine.

14. The composition of claim 1, wherein said composition comprises one or more vitamins.

15. The composition of claim 1, wherein said composition comprises niacinamide, sodium starch octenylsuccinate, calcium pantothenate, maltodextrin, sodium ascorbyl phosphate, tocopheryl acetate, pyridoxine HCl, silica, or a combination thereof.

16. The composition of claim 1, wherein said composition comprises a zwitterionic surfactant and an anionic surfactant in a ratio from about 1:1 to about 1:2.

17. The composition of claim 1, wherein said composition comprises a fatty alcohol and a quaternary nitrogen in a ratio from about 1:1 to about 3:1.

18. A hair care composition comprising between about 0.01 percent and about 5 percent of a quaternized polysiloxane and between about 0.4 percent and about 2 percent of a hydrogenated polydecene.

19. The composition of claim 18, wherein said composition is a conditioner.

20. The composition of claim 18, wherein said composition comprises between about 0.01 percent and about 1.5 percent of said quaternized polysiloxane.

21. The composition of claim 18, wherein said composition comprises between about 0.1 percent and about 1.5 percent of said quaternized polysiloxane.

22. The composition of claim 18, wherein said quaternized polysiloxane is silicone quaternium-12 or 22.

23. The composition of claim 18, wherein said composition comprises one or more botanical compounds.

24. The composition of claim 18, wherein said composition comprises a sunflower seed extract, a sandalwood extract, a Phellodendron amurense bark extract, a barley extract, or a combination thereof.

25. The composition of claim 18, wherein said composition comprises one or more amino acids.

26. The composition of claim 18, wherein said composition comprises creatine.

27. The composition of claim 18, wherein said composition comprises one or more vitamins.

28. The composition of claim 18, wherein said composition comprises niacinamide, sodium starch octenylsuccinate, calcium pantothenate, maltodextrin, sodium ascorbyl phosphate, tocopheryl acetate, pyridoxine HCl, silica, or a combination thereof.

29. The composition of claim 18, wherein said composition comprises a fatty alcohol and a quaternary nitrogen in a ratio from about 1:1 to about 3:1.

30. A hair care composition comprising the ingredients set forth in a table selected from the group consisting of Table 1, 2, 3, 4, 5, 6, 7, 8, 9, 10, 11, 12, 13, 14, 15, 16, 17, 18, 19, and 20, wherein each of said ingredients of said hair care composition is present within said hair care composition at a percentage that is within 2 percent of the percentage value set forth in said table.

31. The composition of claim 30, wherein each of said ingredients of said hair care composition is present within said hair care composition at a percentage that is within 1 percent of the percentage value set forth in said table.

32. The composition of claim 30, wherein each of said ingredients of said hair care composition is present within said hair care composition at a percentage that is within 0.5 percent of the percentage value set forth in said table.

33. The composition of claim 30, wherein each of said ingredients of said hair care composition is present within said hair care composition at a percentage that is within 0.25 percent of the percentage value set forth in said table.

34. The composition of claim 30, wherein each of said ingredients of said hair care composition is present within said hair care composition at the percentage value set forth in said table.
